SEQUENCE LISTING (66 sequences)

SEQ ID NO: 1 - protein, 463 aa, Artificial sequence
  Modified Homo sapiens immunoglobulin G1 heavy chain
MNLLLILTFVAAAVAQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSV

SEQ ID NO: 3 - protein, 234 aa, Artificial sequence
  Modified Homo sapiens immunoglobulin G1 kappa light chain
MNLLLILTFVAAAVADVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 113 aa, Mus musculus
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKR

SEQ ID NO: 5 - protein, 327 aa, Equus caballus
ASTTAPKVFPLASHSAATSGSTVALGCLVSSYFPEPVTVSWNSGALTSGVHTFPSVLQSSGLYSLSSMVTVPASSLKSQTYICNVAHPASSTKVDKKIVIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 6 - protein, 415 aa, Equus caballus
SLEDTAVIPLFSECKAPKEDDVVSLACLVKGYFPEPVQVTWEPEMQNQKPWTFPAMKKGQEYIHVFSLTTWWKPGSHSCTVHHKASSFRKKMTFQEPASWAPQRTSALPVTSKEPTPAPTTLRKSEPSTRHTQPETQKPRIPVDTPLKECQSHTHPPSIYLLHPPLQGLWLKGEATFTCLVVGDDLKDAHLSWELSERSNGMFVESGPLEKHTNGSQSRSSRLALPRSSWAMGTSVTCKLSYPNLLSSMEVVGLKEHAASAPRSLTVHALTTPGLNASPGATSWLQCKVSGFSPPEIVLTWLEGQREVDPSWFATARPTAQPGNTTFQTWSILLVPTIPGPPTATYTCVVGHEASRQLLNTSWSLDTGGLAMTPESKDENSDDYADLDDAGSLWLTFMALFLITLLYSGFVTFIK

SEQ ID NO: 7 - protein, 334 aa, Canis familiaris
SKTSPSVFPLSLCHQESEGYVVIGCLVQGFFPPEPVNVTWNAGKDSTSVKNFPPMKAATGSLYTMSSQLTLPAAQCPDDSSVKCQVQHASSPSKAVSVPCKDNSHPCHPCPSCNEPRLSLQKPALEDLLLGSNASLTCTLSGLKDPKGATFTWNPSKGKEPIQKNPERDSCGCYSVSSVLPGCADPWNHGDTFSCTATHPESKSPITVSITKTTEHIPPQVHLLPPPSEELALNELVTLTCLVRGFKPKDVLVRWLQGTQELPQEKYLTWEPLKEPDQTNMFAVTSMLRVTAEDWKQGEKFSCMVGHEALPMSFTQKTIDRLAGKPTHVNVSVV

SEQ ID NO: 8 - protein, 426 aa, Canis familiaris
TSQDLSVFPLASCCKDNIASTSVTLGCLVTGYLPMSTTVTWDTGSLNKNVTTFPTTFHETYGLHSIVSQVTASGKWAKQRFTCSVAHAESTAINKTFSACALNFIPPTVKLFHSSCNPVGDTHTTIQLLCLISGYVPGDMEVIWLVDGQKATNIFPYTAPGTKEGNVTSTHSELNITQGEWVSQKTYTCQVTYQGFTFKDEARKCSESDPRGVTSYLSPPSPLDLYVHKAPKITCLVVDLATMEGMNLTWYRESKEPVNPGPLNKKDHFNGTITVTSTLPVNTNDWIEGETYYCRVTHPHLPKDIVRSIAKAPGKRAPPDVYLFLPPEEEQGTKDRVTLTCLIQNFFPADISVQWLRNDSPIQTDQYTTTGPHKVSGSRPAFFIFSRLEVSRVDWEQKNKFTCQVVHEALSGSRILQKWVSKTPGK

SEQ ID NO: 9 - protein, 335 aa, Felis catus
ASTTASSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 10 - protein, 96 aa, Camelus dromedarius
EVQLVESGGGLVQPGGSLRLSCAASGFTFSSYDMSWVRQAPGREREGVAAINSGGGSTYYADSVKGRFTISQDNAKNTVYLQMNSLKPEDTAMYYC

SEQ ID NO: 11 - protein, 96 aa, Camelus dromedarius
EVQLVESGGGLVQPGGSLRLSCAASGFTFSSYWMYWVRQAPGKGLEWVSTINSGGGSTYYADSVKGRFTISRDNAKNMLYLQMNSLKPEDTAMYYC

SEQ ID NO: 12 - DNA, 1434 bp, Artificial sequence
  Murine anti-AGE IgG2b heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctaagaccacccctccctccgtgtaccctctggctcctggctgtggcgacaccaccggatcctctgtgaccctgggctgcctcgtgaagggctacttccctgagtccgtgaccgtgacctggaactccggctccctgtcctcctccgtgcacacctttccagccctgctgcagtccggcctgtacaccatgtcctccagcgtgacagtgccctcctccacctggccttcccagaccgtgacatgctctgtggcccaccctgcctcttccaccaccgtggacaagaagctggaaccctccggccccatctccaccatcaacccttgccctccctgcaaagaatgccacaagtgccctgcccccaacctggaaggcggcccttccgtgttcatcttcccacccaacatcaaggacgtgctgatgatctccctgacccccaaagtgacctgcgtggtggtggacgtgtccgaggacgaccctgacgtgcagatcagttggttcgtgaacaacgtggaagtgcacaccgcccagacccagacacacagagaggactacaacagcaccatcagagtggtgtctaccctgcccatccagcaccaggactggatgtccggcaaagaattcaagtgcaaagtgaacaacaaggacctgcccagccccatcgagcggaccatctccaagatcaagggcctcgtgcgggctccccaggtgtacattctgcctccaccagccgagcagctgtcccggaaggatgtgtctctgacatgtctggtcgtgggcttcaaccccggcgacatctccgtggaatggacctccaacggccacaccgaggaaaactacaaggacaccgcccctgtgctggactccgacggctcctacttcatctactccaagctgaacatgaagacctccaagtgggaaaagaccgactccttctcctgcaacgtgcggcacgagggcctgaagaactactacctgaagaaaaccatctcccggtcccccggctag

SEQ ID NO: 13 - DNA, 1416 bp, Artificial sequence
  Chimeric anti-AGE human IgG1 antibody heavy chain
atggaccccaagggcagcctgagctggagaatcctgctgttcctgagcctggccttcgagctgagctacggccaggtgcagctgctgcagccaggtgccgagctcgtgaaacctggcgcctctgtgaagctggcctgcaaggcttccggctacctgttcaccacctactggatgcactggctgaagcagaggccaggccagggcctggaatggatcggcgagatctcccccaccaacggcagagcctactacaacgcccggttcaagtccgaggccaccctgaccgtggacaagtcctccaacaccgcctacatgcagctgtcctccctgacctctgaggcctccgccgtgtactactgcgccagagcttacggcaactacgagttcgcctactggggccagggcaccctcgtgacagtgtctgtggctagcaccaagggccccagcgtgttccctctggcccccagcagcaagagcaccagcggcggaaccgccgccctgggctgcctggtgaaggactacttccccgagcccgtgaccgtgtcctggaacagcggcgctctgaccagcggagtgcacaccttccctgccgtgctgcagagcagcggcctgtactccctgagcagcgtggtgaccgtgcccagcagcagcctgggcacccagacctacatctgcaacgtgaaccacaagccctccaacaccaaggtggacaagaaggtggagcctaagagctgcgacaagacccacacctgccctccctgccccgcccccgagctgctgggcggacccagcgtgttcctgttccctcccaagcccaaggacaccctgatgatcagccgcacccccgaggtgacctgcgtggtggtggacgtgagccacgaggaccccgaggtgaagttcaactggtacgtggacggcgtggaggtgcacaacgccaagaccaagcctcgggaggagcagtacaactccacctaccgcgtggtgagcgtgctgaccgtgctgcaccaggactggctgaacggcaaggagtacaagtgcaaggtgagcaacaaggccctgcccgctcccatcgagaagaccatcagcaaggccaagggccagccccgggagcctcaggtgtacaccctgccccccagccgcgacgagctgaccaagaaccaggtgagcctgacctgcctggtgaagggcttctacccctccgacatcgccgtggagtgggagagcaacggccagcctgagaacaactacaagaccacccctcccgtgctggacagcgacggcagcttcttcctgtacagcaagctgaccgtggacaagtcccggtggcagcagggcaacgtgttcagctgcagcgtgatgcacgaggccctgcacaaccactacacccagaagagcctgagcctgagccccggatag

SEQ ID NO: 14 - DNA, 720 bp, Artificial sequence
  Murine anti-AGE Kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 15 - DNA, 720 bp, Artificial sequence
  Chimeric anti-AGE human kappa light chain
atggagaccgacaccctgctgctctgggtgctgctgctctgggtgcccggctccaccggagacgtcgtgatgacccagacccctctgtccctgcctgtgtctctgggcgaccaggcctccatctcctgccggtctagacagtccctcgtgaactccaacggcaacaccttcctgcagtggtatctgcagaagcccggccagtcccccaagctgctgatctacaaggtgtccctgcggttctccggcgtgcccgacagattttccggctctggctctggcaccgacttcaccctgaagatctcccgggtggaagccgaggacctgggcctgtacttctgcagccagtccacccacgtgccccctacatttggcggaggcaccaagctggaaatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 16 - protein, 477 aa, Artificial sequence
  Murine anti-AGE IgG2b heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPG

SEQ ID NO: 17 - protein, 471 aa, Artificial sequence
  Chimeric anti-AGE human IgG1 heavy chain
MDPKGSLSWRILLFLSLAFELSYGQVQLLQPGAELVKPGASVKLACKASGYLFTTYWMHWLKQRPGQGLEWIGEISPTNGRAYYNARFKSEATLTVDKSSNTAYMQLSSLTSEASAVYYCARAYGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 239 aa, Artificial sequence
  Murine anti-AGE kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19 - protein, 239 aa, Artificial sequence
  Chimeric anti-AGE human kappa light chain
METDTLLLWVLLLWVPGSTGDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 118 aa, Artificial sequence; identical to SEQ ID NO: 2
  Murine anti-AGE IgG2b heavy chain (variable region)

SEQ ID NO: 21 - protein, 112 aa, Artificial sequence
  Murine anti-AGE kappa light chain (variable region)
DVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIK

SEQ ID NO: 22 - protein, 326 aa, Artificial sequence
  Human constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 23 - protein, 7 aa, Artificial sequence
  CDR1H (heavy chain)
SYTMGVS

SEQ ID NO: 24 - protein, 17 aa, Artificial sequence
  CDR2H (heavy chain)
TISSGGGSTYYPDSVKG

SEQ ID NO: 25 - protein, 10 aa, Artificial sequence
  CDR3H (heavy chain)
  misc_feature (10)..(10): Xaa can be any naturally occurring amino acid
QGGWLPPFAX

SEQ ID NO: 26 - protein, 17 aa, Artificial sequence
  CDR1L (light chain)
RASKSVSTSSRGYSYMH

SEQ ID NO: 27 - protein, 7 aa, Artificial sequence
  CDR2L (light chain)
LVSNLES

SEQ ID NO: 28 - protein, 9 aa, Artificial sequence
  CDR3L (light chain)
QHIRELTRS

SEQ ID NO: 29 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNQKFQGRVTMTVDKSTNTVYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaacagaaattccagggcagagtgaccatgaccgtggacaagtccaccaacaccgtgtacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 31 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSTNTAYMELSSLRSEDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccaccaacaccgcttacatggaactgtcctccctgcggagcgaggacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 33 - protein, 468 aa, Artificial Sequence
  Humanized heavy chain
MDPKGSLSWRILLFLSLAFELSYGQVQLVQSGAEVKKPGASVKVSCKASGYLFTTYWMHWVRQAPGQGLEWMGEISPTNGRAYYNAKFQGRVTMTVDKSINTAYMELSRLRSDDTAVYYCARAYGNYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - DNA, 1408 bp, Artificial Sequence
  Humanized heavy chain
atggaccccaagggcagcctgagctggagaatcctgctgttcctgagcctggccttcgagctgagctacggccaggtgcagctggtgcagtctggcgccgaagtgaagaaacctggcgcctccgtgaggtgtcctgcaaggcttccggctacctgttcaccacctactggatgcactgggtgcgacaggcccctggacagggcctggaatggatgggcgagatctcccctaccaacggcagagcctactacaaccaaaattccagggcagagtgaccatgaccgtggacaagtccatcaacaccgcttacatggaactgtccagactgcggagcgatgacaccgccgtgtactactgcgctagagcctacggcaactacgattcgcctactggggccagggcaccctcgtgacagtgtcctctgctagcaccaagggccccagcgtgttccctctggcccccagcagcaagagcaccagcggcggaaccgccgccctgggctgcctgggaaggactacttccccgagcccgtgaccgtgtcctggaacagcggcgctctgaccagcggagtgcacaccttccctgccgtgctgcagagcagcggcctgtactccctgagcagcgtggtgaccgtgccagcagcagcctgggcacccagacctacatctgcaacgtgaaccacaagccctccaacaccaaggtggacaagaaggtggagcctaagagctgcgacaagacccacacctgccctccctgccccgccccgagctgctgggcggacccagcgtgttcctgttccctcccaagcccaaggacaccctgatgatcagccgcacccccgaggtgacctgcgtggtggtggacgtgagccacgaggaccccgaggtgagttcaactggtacgtggacggcgtggaggtgcacaacgccaagaccaagcctcgggaggagcagtacaactccacctaccgcgtggtgagcgtgctgaccgtgctgcaccaggactggctgaacggcaggagtacaagtgcaaggtgagcaacaaggccctgcccgctcccatcgagaagaccatcagcaaggccaagggccagccccgggagcctcaggtgtacaccctgccccccagccgcgacgagctgacaagaaccaggtgagcctgacctgcctggtgaagggcttctacccctccgacatcgccgtggagtgggagagcaacggccagcctgagaacaactacaagaccacccctcccgtgctggacagcgacgcagcttcttcctgtacagcaagctgaccgtggacaagtcccggtggcagcagggcaacgtgttcagctgcagcgtgatgcacgaggccctgcacaaccactacacccagaagagcctgagcctgagcccggatagtaa

SEQ ID NO: 35 - protein, 238 aa, Artificial Sequence
  Humanized light chain
METDTLLLWVLLLWVPGSTGDVVMTQSPLSLPVTLGQPASISCRSSQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatcctcccagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 37 - protein, 238 aa, Artificial Sequence
  Humanized light chain
METDTLLLWVLLLWVPGSTGDVVMTQSPLSLPVTLGQPASISCRSRQSLVNSNGNTFLQWYQQRPGQSPRLLIYKVSLRFSGVPDRFSGSGSGTDFTLKISRVEAEDVGVYYCSQSTHVPPTFGGGTVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 715 bp, Artificial Sequence
  Humanized light chain
atggagaccgacaccctgctgctctgggtgctgctgctctgggtgcccggctccaccggagacgtcgtgatgacccagtcccctctgtccctgcctgtgaccctgggacagcctgcctccatctcctcagatccaggcagtccctcgtgaactccaacggcaacaccttcctgcagtggtatcagcagcggcctggccagagccccagactgctgatctacaaggtgtccctgcggttctccggcgtgcccgacgattttccggctctggctctggcaccgacttcaccctgaagatctcccgggtggaagccgaggacgtgggcgtgtactactgctcccagagcacccacgtgccccctacatttggcggaggcaccaagtggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 39 - protein, 238 aa, Artificial Sequence
  Humanized light chain
METDTLLLWVLLLWVPGSTGDVVMTQSPLSSPVTLGQPASISCRSSQSLVNSNGNTFLQWYHQRPGQPPRLLIYKVSLRFSGVPDRFSGSGAGKDFTLKISRVEAEDVGVYYCSQSTHVPPTFGQGTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 715 bp, Artificial Sequence
  Humanized light chain
atggagaccgacaccctgctgctctgggtgctgctgctctgggtgcccggctccaccggagacgtcgtgatgacccagtcccctctgtccagtcctgtgaccctgggacagcctgcctccatctcctcagatcctcccagtccctcgtgaactccaacggcaacaccttcctgcagtggtatcaccagcggcctggccagcctcccagactgctgatctacaaggtgtccctgcggttctccggcgtgcccgacgattttccggctctggcgctggcaaggacttcaccctgaagatctcccgggtggaagccgaggacgtgggcgtgtactactgctcccagagcacccacgtgccccctacatttggccagggcaccaactggaaatcaagcggaccgtggccgcccccagcgtgttcatcttccctcccagcgacgagcagctgaagtctggcaccgccagcgtggtgtgcctgctgaacaacttctacccccgcgaggccaagggcagtggaaggtggacaacgccctgcagagcggcaacagccaggagagcgtgaccgagcaggactccaaggacagcacctacagcctgagcagcaccctgaccctgagcaaggccgactacgagaagacaaggtgtacgcctgcgaggtgacccaccagggactgtctagccccgtgaccaagagcttcaaccggggcgagtgctaa

SEQ ID NO: 41 - protein, 5 aa, Mus musculus
TYWMH

SEQ ID NO: 42 - protein, 17 aa, Mus musculus
EISPTNGRAYYNARFKS

SEQ ID NO: 43 - protein, 9 aa, Mus musculus
AYGNYEFAY

SEQ ID NO: 44 - protein, 16 aa, Mus musculus
RSRQSLVNSNGNTFLQ

SEQ ID NO: 45 - protein, 7 aa, Mus musculus
KVSLRFS

SEQ ID NO: 46 - protein, 9 aa, Mus musculus
SQSTHVPPT

SEQ ID NO: 47 - protein, 467 aa, Mus musculus
MGWTLVFLFLLSVTAGVHSQVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 467 aa, Artificial sequence
  Humanized heavy chain
MGWTLVFLFLLSVTAGVHSEVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 467 aa, Artificial sequence
  Humanized heavy chain
MGWTLVFLFLLSVTAGVHSQVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 467 aa, Artificial sequence
  Humanized heavy chain
MGWTLVFLFLLSVTAGVHSQVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 467 aa, Artificial sequence
  Humanized heavy chain
MGWTLVFLFLLSVTAGVHSQVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 118 aa, Mus musculus
QVQLLQPGAELVKPGASVKLACKASGYLFTTYWMHWLKQRPGQGLEWIGEISPTNGRAYYNARFKSEATLTVDKSSNTAYMQLSSLTSEASAVYYCARSFGNYEFAYWGQGTLVTVSV

SEQ ID NO: 53 - protein, 118 aa, Artificial sequence
  Humanized heavy chain variable region
EVQLLESGAEAKKPGASVKLSCKASGYLFTTYWMHWVHQAPGQRLEWMGEISPTNGRAYYNARFKSRVTITVDKSASTAYMELSSLRSEDTAVYYCARSFGNYEFAYWGQGTLVTVSS

SEQ ID NO: 54 - protein, 118 aa, Artificial sequence
  Humanized heavy chain variable region
QVQLVQSGAEVKKPGASVKVSCKASGYLFTTYWMHWVRQAPGQRLEWIGEISPTNGRAYYNARFKSRVTITRDTSASTAYMELSSLRSEDTAVYYCARSFGNYEFAYWGQGTLVTVSS

SEQ ID NO: 55 - protein, 118 aa, Artificial sequence
  Humanized heavy chain variable region
QVQLVQSGAEVKKPGSSVKVSCKASGYLFTTYWMHWVRQAPGQGLEWMGEISPTNGRAYYNARFKSRVTITADKSTSTAYMELSSLRSEDTAVYYCARSFGNYEFAYWGQGTLVTVSS

SEQ ID NO: 56 - protein, 118 aa, Artificial sequence
  Humanized heavy chain variable region
QVQLVQSGAEVKKPGASVKVSCEASGYLFTTYWMHWVRQAPGQGLEWMGEISPTNGRAYYNARFKSRVTITRDTSINTAYMELSRLRSDDTAVYYCARSFGNYEFAYWGQGTLVTVSS

SEQ ID NO: 57 - protein, 239 aa, Mus musculus
MVSSAQFLGLLLLCFQGTRCDVVMTQTPLSLPVSLGDQASISCRSRQSLVNSNGNTFLQWYLQKPGQSPKLLIYKVSLRFSGVPDRFSGSGSGTDFTLKISRVEAEDLGLYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 239 aa, Artificial sequence
  Humanized light chain
MVSSAQFLGLLLLCFQGTRCDIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 239 aa, Artificial sequence
  Humanized light chain
MVSSAQFLGLLLLCFQGTRCDIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 239 aa, Artificial sequence
  Humanized light chain
MVSSAQFLGLLLLCFQGTRCDVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 239 aa, Artificial sequence
  Humanized light chain
MVSSAQFLGLLLLCFQGTRCDIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 112 aa, Mus musculus; identical to SEQ ID NO: 21

SEQ ID NO: 63 - protein, 112 aa, Artificial sequence
  Humanized light chain variable region
DIVMTQTPLSLPVTLGQPASISCRSRQSLVNSNGNTFLQWLQQRPGQPPRLLIYKVSLRFSGVPDRFSGSGAGTDFTLTISRVEAEDVGIYFCSQSTHVPPTFGQGTKVEIK

SEQ ID NO: 64 - protein, 112 aa, Artificial sequence
  Humanized light chain variable region
DIVMTQTPLSLSVTPGQPASISCRSRQSLVNSNGNTFLQWYLQKPGQSPQLLIYKVSLRFSGVPDRFSGSGSGTDFTLKISRVEPEDVGVYYCSQSTHVPPTFGGGTKVEVK

SEQ ID NO: 65 - protein, 112 aa, Artificial sequence
  Humanized light chain variable region
DVVMTQSPLSLPVTLGQPASISCRSRQSLVNSNGNTFLQWFQQRPGQSPRRLIYKVSLRFSGVPDRFSGSGSDTDFTLRISRVEAEDVGLYYCSQSTHVPPTFGQGTKLEIK

SEQ ID NO: 66 - protein, 112 aa, Artificial sequence
  Humanized light chain variable region
DIVMTQTPLSLSVTPGQPASISCRSRQSLVNSNGNTFLQWLLQKPGQPPQLLIYKVSLRFSGVPNRFSGSGSGTDFTLKISRVEAEDVGLYYCSQSTHVPPTFGGGTKVEIK

(19) United States
(12) Patent Application Publication
(10) Pub. No.: US 2022/0175916 A1
(43) Pub. Date: Jun. 9, 2022

(54) METHODS AND COMPOSITIONS FOR TREATING CHRONIC EFFECTS OF RADIATION AND CHEMICAL EXPOSURE

(71) Applicant: Siwa Corporation, Chicago, IL (US)

(72) Inventor: Lewis S. Gruber, Chicago, IL (US)

(21) Appl. No.: 17/262,684

(22) PCT Filed: Jul. 23, 2019
(86) PCT No.: PCT/US2019/043071

§ 371 Date: Jan. 22, 2021

Related U.S. Application Data
(60) Provisional application No. 62/702,229, filed on Jul. 23, 2018.

(51) Int. Cl. A61K 39/395; A61P 35/04; C07K 16/44

(52) U.S. Cl. CPC A61K 39/395; A61K 2039/505; C07K 16/44; A61P 35/04

(57) ABSTRACT

A method of treating a chronic effect of radiation or chemical exposure comprises administering to a subject a composition comprising an anti-AGE antibody. A composition for treating a chronic effect of radiation or chemical exposure comprises a first anti-AGE antibody, a second anti-AGE antibody and a pharmaceutically acceptable carrier. The first anti-AGE antibody is different from the second anti-AGE antibody. A method of treating or preventing the onset of a chronic effect of radiation or chemical exposure comprises immunizing a subject in need thereof against AGE-modified proteins or peptides of a cell.

### BACKGROUND

Premature or accelerated aging occurs when an organism exhibits physiological changes that are typically observed in similar organisms having a greater chronological age. Premature aging is a whole-body or systemic condition affecting the entire organism. Some changes may be purely cosmetic, such as the development of gray hair or wrinkles, and have no negative effect on health. Other changes may severely impact physical health, such as the development of cataracts, arteriosclerosis or Alzheimer's disease. In its most severe form, premature aging may result in a shortened lifespan.

Premature aging is a common symptom of the class of genetic disorders known as progeroid syndromes. Most progeroid syndromes are thought to be caused by mutations of a single gene that lead to defects in the DNA repair mechanism or defects in the lamin NC protein (“Progeroid syndromes”, available online at en.wikipedia.org/wiki/Progeroid_syndromes (Nov. 29, 2017)). Examples of progeroid syndromes include Hutchinson-Gilford progeria syndrome (also known as progeria), Werner syndrome, Bloom syndrome, Rothmund-Thomson syndrome, Cockayne syndrome, xeroderma pigmentosum, trichothiodystrophy, combined xeroderma pigmentosum-Cockayne syndrome and restrictive dermopathy. Although the specific mechanisms may vary, these genetic disorders often result in a shortened lifespan. For example, Hutchinson-Gilford progeria syndrome causes accelerated vascular aging, which typically results in premature death due to cardiovascular disease (Ribas, J. et al., “Biomechanical strain exacerbates inflammation on a progeria-on-a-chip model”, Small, Vol. 13 (2017)).

Symptoms which mimic premature aging may be a chronic effect of exposure to certain substances. These symptoms may result from environmental exposure, especially exposure to radiation, and exposure to various chemicals. Unlike premature aging, symptoms which mimic premature aging are typically localized to the area of exposure.

Radiation exposure, particularly exposure to ionizing radiation and ultraviolet (UV) radiation, is a known cause of symptoms which mimic premature aging. Ionizing radiation exposure has been associated with symptoms which mimic premature aging since the 1940s and is known to cause an increase in cancer, cardiovascular disease, dementia and Type II diabetes (Richardson, R. B., “Ionizing radiation and aging: rejuvenating an old idea”, Aging, Vol. 1, No. 11, pp. 887-902 (2009)). Radiotherapy (RT) is often included as part of a cancer treatment regimen and is known to cause considerable long-term damage to healthy tissue, such as the development of pulmonary fibrosis in patients who receive radiotherapy for thoracic-region tumors (Haddadi, G. H. et al., “Hesperidin as radioprotector against radiation-induced lung damage in rat: a histopathological study”, Journal of Medical Physics, Vol. 42, No. 1, pp. 25-32 (2017)). Prolonged unprotected exposure to ultraviolet radiation will cause symptoms which mimic premature aging in skin, including loss of elasticity, loss of pigmentation and degradation of skin texture (Flament, F. et al., “Effect of the sun on visible clinical signs of aging in Caucasian skin”, Clinical, Cosmetic and Investigational Dermatology, Vol. 6, pp. 221-232 (2016)). Radiation exposure combined with other forms of injury, such as burns, blast injury, wounds, blast trauma and infectious complications, results in increased physical harm, especially the combination of radiation and burn injury (Palmer, J. L. et al., “Combined radiation and burn injury results in exaggerated early pulmonary inflammation”, Radiation Research, Vol. 180, No. 3, pp. 276-283 (2013)).

Exposure to chemicals as part of the course of treatment for certain diseases and disorders can also cause symptoms which mimic premature aging. A known side effect of chemotherapy is the development of symptoms which mimic premature aging. Cancer survivors have an earlier onset and higher incidence of endocrinopathies, cardiac dysfunction, osteoporosis, pulmonary fibrosis, secondary cancers and frailty as compared to the general population (Cupit-Link, M. C. et al., “Biology of premature ageing in survivors of cancer”, ESMO Open, Vol. 2, No. e000250, pp. 1-9 (2017)). The human immunodeficiency virus (HIV) treatment regimen known as highly active antiretroviral therapy (HAART) significantly extends the lifespan of HIV-infected patients as compared to untreated HIV-infected patients. However, HAART-treated patients have a reduced life expectancy as compared to the normal population as well as an increased prevalence of cardiovascular disease, diabetes, osteoporosis, kidney and liver disease, metabolic disorders, lipodystrophy, Alzheimer's disease and Parkinson's disease (Smith, R. L. et al., “Premature and accelerated aging: HIV or HAART?”, Frontiers in Genetics, Vol. 3, Article 328, pp. 1-10 (2013)).

Symptoms which mimic premature aging are also a side effect of exposure to chemical agents that cause harm, such as chemical weapons (also known as chemical warfare agents or CWAs) or poisons. Examples of chemical weapons include chlorine gas, phosgene gas, mustard gas (also referred to as sulfur mustard or by its formulation, such as H, HD, HT, HL or HQ), the G-series nerve agents including GA (tabun), GB (sarin), GD (soman) and GF (cyclosarin), the V-series nerve agents including VE, VG, VM, VR and VX, Novichok agents, carbamates and insecticides. Survivors of sulfur mustard (mustard gas) exposure have been found to experience an increase in neuropathic, pulmonary, cardiac, carcinogenic and hematologic complications (Rohani, A. et al., “A case control study of cardiovascular health in chemical war disabled Iranian victims”, Indian Journal of Critical Care Medicine, Vol. 14, No. 3, pp. 109-112 (2010)). Viktor Yushchenko was the victim of a well-known case of dioxin poisoning, which causes symptoms which mimic premature aging including cardiovascular disease, cancer, diabetes and early menopause (White, S. S. et al., “An overview of the effects of dioxins and dioxin-like compounds on vertebrates, as documented in human and ecological epidemiology”, Journal of Environmental Science and Health. Part C, Environmental Carcinogenesis & Ecotoxicology Reviews, Vol. 27, No. 4, pp. 197-211 (2009)). Lead and cadmium poisoning can lead to cardiovascular disease, chronic kidney disease and other aging-related diseases (Zota, A. R. et al., “Associations of cadmium and lead exposure with leukocyte telomere length: findings from national health and nutrition examination survey, 1999-2002”, American Journal of Epidemiology, Vol. 181, No. 2, pp. 127-136 (2015)). Exposure to oxidizing substances can also result in symptoms which mimic premature aging. Treatment of human chondrocytes with hydrogen peroxide in vitro accelerated the aging process (Brandl, A. et al., “Oxidative stress induces senescence in chondrocytes”, Journal of Orthopaedic Research, Vol. 29, pp. 1114-1120 (2011)).

On a cellular level, premature aging may be viewed as an early onset of cellular senescence. Senescent cells are cells that are partially-functional or non-functional and are in a state of proliferative arrest. Senescence is a distinct state of a cell, and is associated with biomarkers, such as activation of the biomarker p16^{Ink4a}, and expression of β-galactosidase. Replicative senescence results from telomere shortening that leads to DNA damage response. Senescence may also be caused by damage or stress (such as overstimulation by growth factors) of cells.

Advanced glycation end-products (AGES; also referred to as AGE-modified proteins, or glycation end-products) arise from a non-enzymatic reaction of sugars with protein side-chains (Ando, K. et al., Membrane Proteins of Human Erythrocytes Are Modified by Advanced Glycation End Products during Aging in the Circulation, Biochem Biophys Res Commun., Vol. 258, 123, 125 (1999)). This process begins with a reversible reaction between the reducing sugar and the amino group to form a Schiff base, which proceeds to form a covalently-bonded Amadori rearrangement product. Once formed, the Amadori product undergoes further rearrangement to produce AGEs. Hyperglycemia, caused by diabetes mellitus (DM), and oxidative stress promote this post-translational modification of membrane proteins (Lindsey J B, et al., “Receptor For Advanced Glycation End-Products (RAGE) and soluble RAGE (sRAGE): Cardiovascular Implications,” Diabetes Vascular Disease Research, Vol. 6(1), 7-14, (2009)). AGEs may also be formed from other processes. For example, the advanced glycation end product, N^{ε}-(carboxymethyl)lysine, is a product of both lipid peroxidation and glycoxidation reactions. AGEs have been associated with several pathological conditions including diabetic complications, inflammation, retinopathy, nephropathy, atherosclerosis, stroke, endothelial cell dysfunction, and neurodegenerative disorders (Bierhaus A, “AGEs and their interaction with AGE-receptors in vascular disease and diabetes mellitus. I. The AGE concept,” Cardiovasc Res, Vol. 37(3), 586-600 (1998)).

AGE-modified proteins are also a marker of senescent cells. This association between glycation end-product and senescence is well known in the art. See, for example, Gruber, L. (WO 2009/143411, 26 Nov. 2009), Ando, K. et al. (Membrane Proteins of Human Erythrocytes Are Modified by Advanced Glycation End Products during Aging in the Circulation, Biochem Biophys Res Commun., Vol. 258, 123, 125 (1999)), Ahmed, E. K. et al. (“Protein Modification and Replicative Senescence of WI-38 Human Embryonic Fibroblasts” Aging Cells, vol. 9, 252, 260 (2010)), Vlassara, H. et al. (Advanced Glycosylation Endproducts on Erythrocyte Cell Surface Induce Receptor-Mediated Phagocytosis by Macrophages, J. Exp. Med., Vol. 166, 539, 545 (1987)) and Vlassara et al. (“High-affinity-receptor-mediated Uptake and Degradation of Glucose-modified Proteins: A Potential Mechanism for the Removal of Senescent Macromolecules” Proc. Natl. Acad. Sci. USA, Vol. 82, 5588, 5591 (1985)). Furthermore, Ahmed, E. K. et al. indicates that glycation end-products are “one of the major causes of spontaneous damage to cellular and extracellular proteins” (Ahmed, E. K. et al., see above, page 353). Accordingly, the accumulation of glycation end-products is associated with senescence and lack of function.

The damage or stress that causes cellular senescence also negatively impacts mitochondrial DNA in the cells to cause them to produce free radicals which react with sugars in the cell to form methyl glyoxal (MG). MG in turn reacts with proteins or lipids to generate advanced glycation end products. In the case of the protein component lysine, MG reacts to form carboxymethyllysine, which is an AGE.

Damage or stress to mitochondrial DNA also sets off a DNA damage response which induces the cell to produce cell cycle blocking proteins. These blocking proteins prevent the cell from dividing. Continued damage or stress causes mTOR production, which in turn activates protein synthesis and inactivates protein breakdown. Further stimulation of the cells leads to programmed cell death (apoptosis).

p16 is a protein involved in regulation of the cell cycle, by inhibiting the S phase (synthesis phase). It can be activated during ageing or in response to various stresses, such as DNA damage, oxidative stress or exposure to drugs. p16 is typically considered a tumor suppressor protein, causing a cell to become senescent in response to DNA damage and irreversibly preventing the cell from entering a hyperproliferative state. However, there has been some ambiguity in this regard, as some tumors show overexpression of p16, while other show downregulated expression. Evidence suggests that overexpression of p16 is some tumors results from a defective retinoblastoma protein (“Rb”). p16 acts on Rb to inhibit the S phase, and Rb downregulates p16, creating negative feedback. Defective Rb fails to both inhibit the S phase and downregulate p16, thus resulting in overexpression of p16 in hyperproliferating cells (Romagosa, C. et al., p16^{Ink4a} overexpression in cancer: a tumor suppressor gene associated with senescence and high-grade tumors, Oncogene, Vol. 30, 2087-2097 (2011)).

Senescent cells are associated with secretion of many factors involved in intercellular signaling, including pro-inflammatory factors; secretion of these factors has been termed the senescence-associated secretory phenotype, or SASP (Freund, A. “Inflammatory networks during cellular senescence: causes and consequences” Trends Mol Med. 2010 May; 16(5):238-46). Autoimmune diseases, such as Crohn's disease and rheumatoid arthritis, are associated with chronic inflammation (Ferraccioli, G. et al. “Interleukin-1β and Interleukin-6 in Arthritis Animal Models: Roles in the Early Phase of Transition from Acute to Chronic Inflammation and Relevance for Human Rheumatoid Arthritis” Mol Med. 2010 November-December; 16(11-12): 552-557). Chronic inflammation may be characterized by the presence of pro-inflammatory factors at levels higher than baseline near the site of pathology, but lower than those found in acute inflammation. Examples of these factors include TNF, IL-1α, IL-1β, IL-5, IL-6, IL-8, IL-12, IL-23, CD2, CD3, CD20, CD22, CD52, CD80, CD86, C5 complement protein, BAFF, APRIL, IgE, α4β1 integrin and a4137 integrin. Senescent cells also upregulate genes with roles in inflammation including IL-1β, IL-8, ICAM1, TNFAP3, ESM1 and CCL2 (Burton, D. G. A. et al., “Microarray analysis of senescent vascular smooth muscle cells: a link to atherosclerosis and vascular calcification”, Experimental Gerontology, Vol. 44, No. 10, pp. 659-665 (October 2009)).

Senescent cells secrete reactive oxygen species (“ROS”) as part of the SASP. ROS are believed to play an important role in maintaining senescence of cells. The secretion of ROS creates a bystander effect, where senescent cells induce senescence in neighboring cells: ROS create the very cellular damage known to activate p16 expression, leading to senescence (Nelson, G., A senescent cell bystander effect: senescence-induced senescence, Aging Cell, Vo. 11, 345-349 (2012)). The p16/Rb pathway leads to the induction of ROS, which in turn activates the protein kinase C delta creating a positive feedback loop that further enhance ROS, helping maintain the irreversible cell cycle arrest; it has even been suggested that exposing cancer cells to ROS might be effective to treat cancer by inducing cell phase arrest in hyperproliferating cells (Rayess, H. et al., Cellular senescence and tumor suppressor gene p16, Int J Cancer, Vol. 130, 1715-1725 (2012)).

Recent research demonstrates the therapeutic benefits of removing senescent cells. In vivo animal studies at the Mayo Clinic in Rochester, Minn., found that elimination of senescent cells in transgenic mice carrying a biomarker for elimination delayed age-related disorders associated with cellular senescence. Eliminating senescent cells in fat and muscle tissues substantially delayed the onset of sarcopenia and cataracts and reduced senescence indicators in skeletal muscle and the eye (Baker, D. J. et al., “Clearance of p16^{Ink4a}-positive senescent cells delays ageing-associated disorders”, Nature, Vol. 479, pp. 232-236, (2011)). Mice that were treated to induce senescent cell elimination were found to have larger diameters of muscle fibers as compared to untreated mice. Treadmill exercise tests indicated that treatment also preserved muscle function. Continuous treatment of transgenic mice for removal of senescent cells had no negative side effects and selectively delayed age-related phenotypes that depend on cells. This data demonstrates that removal of senescent cells produces beneficial therapeutic effects and shows that these benefits may be achieved without adverse effects.

Additional In vivo animal studies in mice found that removing senescent cells using senolytic agents treats aging-related disorders, atherosclerosis and pulmonary fibrosis. Short-term treatment with senolytic drugs in chronologically aged or progeroid mice alleviated several aging-related phenotypes (Zhu, Y. et al., “The Achilles' heel of senescent cells: from transcriptome to senolytic drugs”, Aging Cell, vol. 14, pp. 644-658 (2015)). Long-term treatment with senolytic drugs improved vasomotor function in mice with established atherosclerosis and reduced intimal plaque calcification (Roos, C. M. et al., “Chronic senolytic treatment alleviates established vasomotor dysfunction in aged or atherosclerotic mice”, Aging Cell (2016)). Removing senescent cells by administering senolytic agents reversed radiation-induced pulmonary fibrosis in mice (Pan, J. et al., “Inhibition of Bcl-2/xl with ABT-263 selectively kills senescent type II pneumocytes and reverses pulmonary fibrosis induced by ionizing radiation in mice”, International Journal of Radiation Oncology Biology Physics, Vol. 99, No. 2, pp. 353-361 (2017)). This data further demonstrates the benefits of removing senescent cells.

Vaccines have been widely used since their introduction by Edward Jenner in the 1770s to confer immunity against a wide range of diseases and afflictions. Vaccine preparations contain a selected immunogenic agent capable of stimulating immunity to an antigen. Typically, antigens are used as the immunogenic agent in vaccines, such as, for example, viruses, either killed or attenuated, and purified viral components. Antigens used in the production of cancer vaccines include, for example, tumor-associated carbohydrate antigens (TACAs), dendritic cells, whole cells and viral vectors. Different techniques are employed to produce the desired amount and type of antigen being sought. For example, pathogenic viruses are grown either in eggs or cells. Recombinant DNA technology is often utilized to generate attenuated viruses for vaccines.

Vaccines may therefore be used to stimulate the production of antibodies in the body and provide immunity against antigens. When an antigen is introduced to a subject that has been vaccinated and developed immunity to that antigen, the immune system may destroy or remove cells that express the antigen.

### SUMMARY

In a first aspect, the invention is a method of treating or preventing the onset of a chronic effect of radiation exposure comprising administering to a subject a composition comprising an anti-AGE antibody.

In a second aspect, the invention is a method of treating or preventing the onset of a chronic effect of radiation exposure comprising administering to a subject a composition comprising a first anti-AGE antibody and a second anti-AGE antibody. The second anti-AGE antibody is different from the first anti-AGE antibody.

In a third aspect, the invention is a method of treating a subject experiencing a chronic effect of radiation exposure comprising a first administering of an anti-AGE antibody; followed by testing the subject for effectiveness of the first administration at treating the chronic effect of radiation exposure; followed by a second administering of the anti-AGE antibody.

In a fourth aspect, the invention is use of an anti-AGE antibody for the manufacture of a medicament for treating or preventing the onset of a chronic effect of radiation exposure.

In a fifth aspect, the invention is a composition comprising an anti-AGE antibody for use in treating or preventing the onset of a chronic effect of radiation exposure.

In a sixth aspect, the invention is a composition for treating or preventing the onset of a chronic effect of radiation exposure comprising a first anti-AGE antibody, a second anti-AGE antibody and a pharmaceutically-acceptable carrier. The first anti-AGE antibody is different from the second anti-AGE antibody.

In a seventh aspect, the invention is a method of treating or preventing the onset of a chronic effect of radiation exposure comprising immunizing a subject in need thereof against AGE-modified proteins or peptides of a cell.

In an eighth aspect, the invention is a method of treating a subject experiencing a chronic effect of radiation exposure comprising administering a first vaccine comprising a first AGE antigen and, optionally, administering a second vaccine comprising a second AGE antigen. The second AGE antigen is different from the first AGE antigen.

In a ninth aspect, the invention is use of an AGE antigen for the manufacture of a medicament for treating or preventing the onset of a chronic effect of radiation exposure.

In a tenth aspect, the invention is a composition comprising an AGE antigen for use in treating or preventing the onset of a chronic effect of radiation exposure.

In an eleventh aspect, the invention is a method of treating or preventing the onset of a chronic effect of chemical exposure comprising administering to a subject a composition comprising an anti-AGE antibody.

In a twelfth aspect, the invention is a method of treating or preventing the onset of a chronic effect of chemical exposure comprising administering to a subject a composition comprising a first anti-AGE antibody and a second anti-AGE antibody. The second anti-AGE antibody is different from the first anti-AGE antibody.

In a thirteenth aspect, the invention is a method of treating a subject experiencing a chronic effect of chemical exposure comprising a first administering of an anti-AGE antibody; followed by testing the subject for effectiveness of the first administration at treating the chronic effect of chemical exposure; followed by a second administering of the anti-AGE antibody.

In a fourteenth aspect, the invention is use of an anti-AGE antibody for the manufacture of a medicament for treating or preventing the onset of a chronic effect of chemical exposure.

In a fifteenth aspect, the invention is a composition comprising an anti-AGE antibody for use in treating or preventing the onset of a chronic effect of chemical exposure.

In a sixteenth aspect, the invention is a composition for treating or preventing the onset of a chronic effect of chemical exposure comprising a first anti-AGE antibody, a second anti-AGE antibody and a pharmaceutically-acceptable carrier. The first anti-AGE antibody is different from the second anti-AGE antibody.

In a seventeenth aspect, the invention is a method of treating or preventing the onset of a chronic effect of chemical exposure comprising immunizing a subject in need thereof against AGE-modified proteins or peptides of a cell.

In an eighteenth aspect, the invention is a method of treating a subject experiencing a chronic effect of chemical exposure comprising administering a first vaccine comprising a first AGE antigen and, optionally, administering a second vaccine comprising a second AGE antigen. The second AGE antigen is different from the first AGE antigen.

In a nineteenth aspect, the invention is use of an AGE antigen for the manufacture of a medicament for treating or preventing the onset of a chronic effect of chemical exposure.

In a twentieth aspect, the invention is a composition comprising an AGE antigen for use in treating or preventing the onset of a chronic effect of chemical exposure.

### Definitions

The term “premature aging” means the development or onset of physiological changes that are typically observed in similar organisms having a greater chronological age. Premature aging is a whole-body or systemic condition that affects the entire organism.

The term “radiation” includes alpha radiation, beta radiation, gamma radiation, X-ray radiation, and neutron radiation.

The term “chronic effect” means an effect that is characterized by symptoms which mimic premature aging.

The term “peptide” means a molecule composed of 2-50 amino acids.

The term “protein” means a molecule composed of more than 50 amino acids.

The terms “advanced glycation end-product”, “AGE”, “AGE-modified protein or peptide” and “glycation end-product” refer to modified proteins or peptides that are formed as the result of the reaction of sugars with protein side chains that further rearrange and form irreversible cross-links. This process begins with a reversible reaction between a reducing sugar and an amino group to form a Schiff base, which proceeds to form a covalently-bonded Amadori rearrangement product. Once formed, the Amadori product undergoes further rearrangement to produce AGEs. AGE-modified proteins and antibodies to AGE-modified proteins are described in U.S. Pat. No. 5,702,704 to Bucala (“Bucala”) and U.S. Pat. No. 6,380,165 to Al-Abed et al. (“Al-Abed”). Glycated proteins or peptides that have not undergone the necessary rearrangement to form AGEs, such as N-deoxyfructosyllysine found on glycated albumin, are not AGEs. AGEs may be identified by the presence of AGE modifications (also referred to as AGE epitopes or AGE moieties) such as 2-(2-furoyl)-4(5)-(2-furanyl)-1H-imidazole (“FFI”); 5-hydroxymethyl-1-alkylpyrrole-2-carbaldehyde (“Pyrraline”); 1-alkyl-2-formyl-3,4-diglycosyl pyrrole (“AFGP”), a non-fluorescent model AGE; carboxymethyllysine; carboxyethyllysine; and pentosidine. ALI, another AGE, is described in Al-Abed.

The term “AGE antigen” means a substance that elicits an immune response against an AGE-modified protein or peptide of a cell. The immune response against an AGE-modified protein or peptide of a cell does not include the production of antibodies to the non-AGE-modified protein or peptide.

“An antibody that binds to an AGE-modified protein on a cell”, “anti-AGE antibody” or “AGE antibody” means an antibody, antibody fragment or other protein or peptide that binds to an AGE-modified protein or peptide which preferably includes a constant region of an antibody, where the protein or peptide which has been AGE-modified is a protein or peptide normally found bound on the surface of a cell, preferably a mammalian cell, more preferably a human, cat, dog, horse, camelid (for example, camel or alpaca), cattle, sheep, pig, or goat cell. “An antibody that binds to an AGE-modified protein on a cell”, “anti-AGE antibody” or “AGE antibody” does not include an antibody or other protein which binds with the same specificity and selectivity to both the AGE-modified protein or peptide, and the same non-AGE-modified protein or peptide (that is, the presence of the AGE modification does not increase binding). AGE-modified albumin is not an AGE-modified protein on a cell, because albumin is not a protein normally found bound on the surface of cells. “An antibody that binds to an AGE-modified protein on a cell”, “anti-AGE antibody” or “AGE antibody” only includes those antibodies which lead to removal, destruction, or death of the cell. Also included are antibodies which are conjugated, for example to a toxin, drug, or other chemical or particle. Preferably, the antibodies are monoclonal antibodies, but polyclonal antibodies are also possible.

The term “senescent cell” means a cell which is in a state of proliferative arrest and expresses one or more biomarkers of senescence, such as activation of p16^{Ink4a} or expression of senescence-associated β-galactosidase. Also included are cells which express one or more biomarkers of senescence, do not proliferate in vivo, but may proliferate in vitro under certain conditions, such as some satellite cells found in the muscles of ALS patients.

The term “senolytic agent” means a small molecule with a molecular weight of less than 900 daltons that destroys senescent cells. The term “senolytic agent” does not include antibodies, antibody conjugates, proteins, peptides or biologic therapies.

The term “variant” means a nucleotide, protein or amino acid sequence different from the specifically identified sequences, wherein one or more nucleotides, proteins or amino acid residues is deleted, substituted or added. Variants may be naturally-occurring allelic variants, or non-naturally-occurring variants. Variants of the identified sequences may retain some or all of the functional characteristics of the identified sequences.

The term “percent (%) sequence identity” is defined as the percentage of amino acid residues in a candidate sequence that are identical to the amino acid residues in a reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using publicly available computer software such as BLAST, BLAST-2, ALIGN or Megalign (DNASTAR) software. Preferably, % sequence identity values are generated using the sequence comparison computer program ALIGN-2. The ALIGN-2 sequence comparison computer program is publicly available from Genentech, Inc. (South San Francisco, Calif.), or may be compiled from the source code, which has been filed with user documentation in the U.S. Copyright Office and is registered under U.S. Copyright Registration No. TXU510087. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. Where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained using the ALIGN-2 computer program.

### BRIEF DESCRIPTION OF THE DRAWINGS

FIG. 1 is a graph of the response versus time in an antibody binding experiment.

FIG. 2A illustrates untreated cells after staining with a senescence β-galactosidase staining kit.

FIG. 2B illustrates untreated cells after staining with an anti-AGE antibody conjugated to GFP.

FIG. 2C illustrates untreated cells after staining with an anti-AGE antibody conjugated to GFP-DAPI.

FIG. 2D illustrates etoposide-treated cells after staining with a senescence β-galactosidase staining kit.

FIG. 2E illustrates etoposide-treated cells after staining with an anti-AGE antibody conjugated to GFP.

FIG. 2F illustrates etoposide-treated cells after staining with an anti-AGE antibody conjugated to GFP-DAPI.

FIG. 3A illustrates the results of treating cells with 0 μM doxorubicin for 3 days.

FIG. 3B illustrates the results of treating cells with 0.01 μM doxorubicin for 3 days.

FIG. 3C illustrates the results of treating cells with 0.1 μM doxorubicin for 3 days.

FIG. 3D illustrates the results of treating cells with 1 μM doxorubicin for 3 days.

FIG. 3E illustrates the results of treating cells with 0 μM doxorubicin for 6 days.

FIG. 3F illustrates the results of treating cells with 0.1 μM doxorubicin for 6 days.

FIG. 3G illustrates the results of treating cells with 1 μM doxorubicin for 6 days.

### DETAILED DESCRIPTION

Recent studies have revealed an association between inflammation and the chronic effects of radiation or chemical exposure, such as symptoms which mimic premature aging. Dioxin poisoning induces inflammation, including the expression of the cytokines TNFα, IL-6 and IL-1β, and causes an increased expression of genes whose products are involved in oxidative stress (White, S. S. et al.). Ultraviolet light induces inflammation of the skin, causing a cascade of cytokines, and generates reactive oxygen species including superoxide anion, hydrogen peroxide and the hydroxyl radical (D'Orazio, J. et al., “UV radiation and the skin”, International Journal of Molecular Sciences, Vol. 14, pp. 12222-12248 (2013)). Ionizing radiation, the antiretroviral drugs involved in HAART therapy, cadmium exposure and lead exposure all contribute to symptoms which mimic premature aging by promoting both oxidative stress and inflammation (Zota, A. R. et al.; Smith, R. L. et al.; Richardson, R. B.; Haddadi, G. H. et al.). This is consistent with studies that have found an association between inflammation and premature aging resulting from progeroid syndromes. For example, an organ-on-a-chip model of progeria showed increased levels of inflammation markers in response to biomechanical strain (Ribas, J. et al., “Biomechanical strain exacerbates inflammation on a progeria-on-a-chip model”, Small, Vol. 13 (2017)).

The role of inflammation and oxidative stress in the chronic effects of radiation or chemical exposure implicates cellular senescence. Senescent cells are known to secrete inflammatory factors and reactive oxygen species as part of the senescence-associated secretory phenotype (SASP). These characteristics suggest that cellular senescence is a causative factor in the chronic effects of radiation or chemical exposure, such as the development or onset of symptoms which mimic premature aging. Evidence supporting this relationship may be found in multiple studies showing that chemotherapeutic drugs and ionizing radiation are direct causes of cellular senescence (Cupit-Link, M. C. et al.; Richardson, R. B.; Roninson, I. B., “Tumor cell senescence in cancer treatment”, Cancer Research, Vol. 63, pp. 2705-2715 (2003); Meng, A. et al., “Ionizing radiation and Bisulfan induce premature senescence in murine bone marrow hematopoietic cells”, Cancer Research, Vol. 63, pp. 5414-5419 (2003)).

Removing senescent cells by administration of senolytic agents has been shown to treat symptoms which mimic premature aging resulting from ionization radiation exposure (Zhu, Y. et al.; Pan, J. et al.). The identification of a common link between cellular senescence and the chronic effects of radiation or chemical exposure allows for similar treatment possibilities that target sources of inflammation and oxidative stress. The present invention uses enhanced clearance of cells expressing AGE-modified proteins or peptides (AGE-modified cells) to treat, ameliorate or prevent the onset of the chronic effects of radiation or chemical exposure, such as symptoms which mimic premature aging. This may be accomplished by administering anti-AGE antibodies to a subject.

Vaccination against AGE-modified proteins or peptides of a cell may also be used to control the presence of AGE-modified cells in a subject. The continuous and virtually ubiquitous surveillance exercised by the immune system in the body in response to a vaccination allows maintaining low levels of AGE-modified cells in the body. Vaccination against AGE-modified proteins or peptides of a cell removes or kills senescent cells. The process of senescent cell removal or destruction allows vaccination against AGE-modified proteins or peptides of a cell to be used to treat or prevent the onset of the chronic effects of radiation or chemical exposure, such as symptoms which mimic premature aging.

Premature aging is characterized by the onset of physiological changes, diseases, disorders and/or conditions that are typically exhibited in organisms with an advanced chronological age. Signs of premature aging include the development of gray hair, wrinkles, frailty, cataracts, arteriosclerosis, atherosclerosis, Alzheimer's disease, Parkinson's disease, sarcopenia, loss of adipose tissue, lordokyphosis, cancer, premature menopause, cardiovascular disease, dementia, Type II diabetes, endocrinopathies, cardiac dysfunction, osteoporosis, osteoarthritis, pulmonary fibrosis, kidney and liver disease, metabolic disorders, lipodystrophy, hearing loss, vision loss and memory loss. Premature aging may result from one or more progeroid syndromes. Symptoms which mimic premature aging may be a chronic effect of environmental exposure, such as exposure to radiation, or exposure to chemicals, such as chemotherapy drugs, HAART drugs, chemical weapons, poisons or oxidizing agents.

Anti-AGE antibodies are known in the art and are commercially available. Examples include those described in U.S. Pat. No. 5,702,704 (Bucala) and U.S. Pat. No. 6,380,165 (AI-Abed et al.). The antibody may bind to one or more AGE-modified proteins or peptides having an AGE modification such as FFI, pyrraline, AFGP, ALI, carboxymethyllysine (CML), carboxyethyllysine (CEL) and pentosidine, and mixtures of such antibodies. Preferably, the antibody is non-immunogenic to the animal in which it will be used, such as non-immunogenic to humans; companion animals including cats, dogs and horses; and commercially important animals, such camels (or alpaca), cattle (bovine), sheep, pig, and goats. More preferably, the antibody has the same species constant region as antibodies of the animal to reduce the immune response against the antibody, such as being humanized (for humans), felinized (for cats), caninized (for dogs), equuinized (for horses), camelized (for camels or alpaca), bovinized (for cattle), ovinized (for sheep), porcinized (for pigs), or caperized (for goats). Most preferably, the antibody is identical to that of the animal in which it will be used (except for the variable region), such as a human antibody, a cat antibody, a dog antibody, a horse antibody, a camel antibody, a bovine antibody, a sheep antibody, a pig antibody, or a goat antibody. Details of the constant regions and other parts of antibodies for these animals are described below. The antibody may be monoclonal or polyclonal. Preferably, the antibody is a monoclonal antibody.

Preferred anti-AGE antibodies include those which bind to proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents). A preferred antibody includes the variable region of the commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, Minn.; catalog no. MAB3247), modified to have a human constant region (or the constant region of the animal into which it will be administered). Commercially-available antibodies, such as the carboxymethyl lysine antibody corresponding to catalog no. MAB3247 from R&D Systems, Inc., may be intended for diagnostic purposes and may contain material that is not suited for use in animals or humans. Preferably, commercially-available antibodies are purified and/or isolated prior to use in animals or humans to remove toxins or other potentially-harmful material.

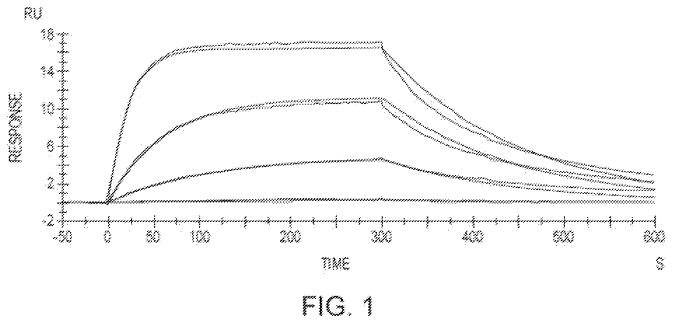

The anti-AGE antibody preferably has a low rate of dissociation from the antibody-antigen complex, or k_{d} (also referred to as k_{back} or off-rate), preferably at most 9×10⁻³, 8×10⁻³, 7×10⁻³ or 6×10⁻³ (sec⁻¹). The anti-AGE antibody preferably has a high affinity for the AGE-modified protein of a cell, which may be expressed as a low dissociation constant K_{D} of at most 9×10⁻⁶, 8×10⁻⁶, 7×10⁻⁶, 6×10⁻⁶, 5×10⁻⁶, 4×10⁻⁶ or 3×10⁻⁶ (M). Preferably, the binding properties of the anti-AGE antibody are similar to, the same as, or superior to the carboxymethyl lysine MAb (Clone 318003) available from R&D Systems, Inc. (Minneapolis, Minn.; catalog no. MAB3247), illustrated in FIG. 1.

The anti-AGE antibody may destroy AGE-modified cells through antibody-dependent cell-mediated cytotoxicity (ADCC). ADCC is a mechanism of cell-mediated immune defense in which an effector cell of the immune system actively lyses a target cell whose membrane-surface antigens have been bound by specific antibodies. ADCC may be mediated by natural killer (NK) cells, macrophages, neutrophils or eosinophils. The effector cells bind to the Fc portion of the bound antibody. The anti-AGE antibody may also destroy AGE-modified cells through complement-dependent cytotoxicity (CDC). In CDC, the complement cascade of the immune system is triggered by an antibody binding to a target antigen.

The anti-AGE antibody may be conjugated to an agent that causes the destruction of AGE-modified cells. Such agents may be a toxin, a cytotoxic agent, magnetic nanoparticles, and magnetic spin-vortex discs.

A toxin, such as pore-forming toxins (PFT) (Aroian R. et al., “Pore-Forming Toxins and Cellular Non-Immune Defenses (CNIDs),” Current Opinion in Microbiology, 10:57-61 (2007)), conjugated to an anti-AGE antibody may be injected into a patient to selectively target and remove AGE-modified cells. The anti-AGE antibody recognizes and binds to AGE-modified cells. Then, the toxin causes pore formation at the cell surface and subsequent cell removal through osmotic lysis.

Magnetic nanoparticles conjugated to the anti-AGE antibody may be injected into a patient to target and remove AGE-modified cells. The magnetic nanoparticles can be heated by applying a magnetic field in order to selectively remove the AGE-modified cells.

As an alternative, magnetic spin-vortex discs, which are magnetized only when a magnetic field is applied to avoid self-aggregation that can block blood vessels, begin to spin when a magnetic field is applied, causing membrane disruption of target cells. Magnetic spin-vortex discs, conjugated to anti-AGE antibodies specifically target AGE-modified cell types, without removing other cells.

Antibodies are Y-shaped proteins composed of two heavy chains and two light chains. The two arms of the Y shape form the fragment antigen-binding (Fab) region while the base or tail of the Y shape forms the fragment crystallizable (Fc) region of the antibody. Antigen binding occurs at the terminal portion of the fragment antigen-binding region (the tips of the arms of the Y shape) at a location referred to as the paratope, which is a set of complementarity determining regions (also known as CDRs or the hypervariable region). The complementarity determining regions vary among different antibodies and give a given antibody its specificity for binding to a given antigen. The fragment crystallizable region of the antibody determines the result of antigen binding and may interact with the immune system, such as by triggering the complement cascade or initiating antibody-dependent cell-mediated cytotoxicity (ADCC). When antibodies are prepared recombinantly, it is also possible to have a single antibody with variable regions (or complementary determining regions) that bind to two different antigens, with each tip of the Y shape being specific to one of the antigens; these are referred to as bi-specific antibodies.

A humanized anti-AGE antibody according to the present invention may have the human constant region sequence of amino acids shown in SEQ ID NO: 22. The heavy chain complementarity determining regions of the humanized anti-AGE antibody may have one or more of the protein sequences shown in SEQ ID NO: 23 (CDR1H), SEQ ID NO: 24 (CDR2H) and SEQ ID NO: 25 (CDR3H). The light chain complementarity determining regions of the humanized anti-AGE antibody may have one or more of the protein sequences shown in SEQ ID NO: 26 (CDR1L), SEQ ID NO: 27 (CDR2L) and SEQ ID NO: 28 (CDR3L).

The heavy chain of a humanized anti-AGE antibody may have or may include the protein sequence of SEQ ID NO: 1. The variable domain of the heavy chain may have or may include the protein sequence of SEQ ID NO: 2. The complementarity determining regions of the variable domain of the heavy chain (SEQ ID NO: 2) are shown in SEQ ID NO: 41, SEQ ID NO: 42 and SEQ ID NO: 43. The kappa light chain of a humanized anti-AGE antibody may have or may include the protein sequence of SEQ ID NO: 3. The variable domain of the kappa light chain may have or may include the protein sequence of SEQ ID NO: 4. Optionally, the arginine (Arg or R) residue at position 128 of SEQ ID NO: 4 may be omitted. The complementarity determining regions of the variable domain of the light chain (SEQ ID NO: 4) are shown in SEQ ID NO: 44, SEQ ID NO: 45 and SEQ ID NO: 46. The variable regions may be codon-optimized, synthesized and cloned into expression vectors containing human immunoglobulin G1 constant regions. In addition, the variable regions may be used in the preparation of non-human anti-AGE antibodies.

The antibody heavy chain may be encoded by the DNA sequence of SEQ ID NO: 12, a murine anti-AGE immunoglobulin G2b heavy chain. The protein sequence of the murine anti-AGE immunoglobulin G2b heavy chain encoded by SEQ ID NO: 12 is shown in SEQ ID NO: 16. The variable region of the murine antibody is shown in SEQ ID NO: 20, which corresponds to positions 25-142 of SEQ ID NO: 16. The antibody heavy chain may alternatively be encoded by the DNA sequence of SEQ ID NO: 13, a chimeric anti-AGE human immunoglobulin G1 heavy chain. The protein sequence of the chimeric anti-AGE human immunoglobulin G1 heavy chain encoded by SEQ ID NO: 13 is shown in SEQ ID NO: 17. The chimeric anti-AGE human immunoglobulin includes the murine variable region of SEQ ID NO: 20 in positions 25-142. The antibody light chain may be encoded by the DNA sequence of SEQ ID NO: 14, a murine anti-AGE kappa light chain. The protein sequence of the murine anti-AGE kappa light chain encoded by SEQ ID NO: 14 is shown in SEQ ID NO: 18. The variable region of the murine antibody is shown in SEQ ID NO: 21, which corresponds to positions 21-132 of SEQ ID NO: 18. The antibody light chain may alternatively be encoded by the DNA sequence of SEQ ID NO: 15, a chimeric anti-AGE human kappa light chain. The protein sequence of the chimeric anti-AGE human kappa light chain encoded by SEQ ID NO: 15 is shown in SEQ ID NO: 19. The chimeric anti-AGE human immunoglobulin includes the murine variable region of SEQ ID NO: 21 in positions 21-132.

A humanized anti-AGE antibody according to the present invention may have or may include one or more humanized heavy chains or humanized light chains. A humanized heavy chain may be encoded by the DNA sequence of SEQ ID NO: 30, 32 or 34. The protein sequences of the humanized heavy chains encoded by SEQ ID NOs: 30, 32 and 34 are shown in SEQ ID NOs: 29, 31 and 33, respectively. A humanized light chain may be encoded by the DNA sequence of SEQ ID NO: 36, 38 or 40. The protein sequences of the humanized light chains encoded by SEQ ID NOs: 36, 38 and 40 are shown in SEQ ID NOs: 35, 37 and 39, respectively. Preferably, the humanized anti-AGE antibody maximizes the amount of human sequence while retaining the original antibody specificity. A complete humanized antibody may be constructed that contains a heavy chain having a protein sequence chosen from SEQ ID NOs: 29, 31 and 33 and a light chain having a protein sequence chosen from SEQ ID NOs: 35, 37 and 39.

Particularly preferred anti-AGE antibodies may be obtained by humanizing murine monoclonal anti-AGE antibodies. Murine monoclonal anti-AGE antibodies have the heavy chain protein sequence shown in SEQ ID NO: 47 (the protein sequence of the variable domain is shown in SEQ ID NO: 52) and the light chain protein sequence shown in SEQ ID NO: 57 (the protein sequence of the variable domain is shown in SEQ ID NO: 62). A preferred humanized heavy chain may have the protein sequence shown in SEQ ID NO: 48, SEQ ID NO: 49, SEQ ID NO: 50 or SEQ ID NO: 51 (the protein sequences of the variable domains of the humanized heavy chains are shown in SEQ ID NO: 53, SEQ ID NO: 54, SEQ ID NO: 55 and SEQ ID NO: 56, respectively). A preferred humanized light chain may have the protein sequence shown in SEQ ID NO: 58, SEQ ID NO: 59, SEQ ID NO: 60 or SEQ ID NO: 61 (the protein sequences of the variable domains of the humanized light chains are shown in SEQ ID NO: 63, SEQ ID NO: 64, SEQ ID NO: 65 and SEQ ID NO: 66, respectively). Preferably, a humanized anti-AGE monoclonal antibody is composed a heavy chain having a protein sequence selected from the group consisting of SEQ ID NO: 48, SEQ ID NO: 49, SEQ ID NO: 50 and SEQ ID NO: 51 and a light chain having a protein sequence selected from the group consisting of SEQ ID NO: 58, SEQ ID NO: 59, SEQ ID NO: 60 and SEQ ID NO: 61. Humanized monoclonal anti-AGE antibodies composed of these protein sequences may have better binding and/or improved activation of the immune system, resulting in greater efficacy.

The protein sequence of an antibody from a non-human species may be modified to include the variable domain of the heavy chain having the sequence shown in SEQ ID NO: 2 or the kappa light chain having the sequence shown in SEQ ID NO: 4. The non-human species may be a companion animal, such as the domestic cat or domestic dog, or livestock, such as cattle, the horse or the camel. Preferably, the non-human species is not the mouse. The heavy chain of the horse (Equus caballus) antibody immunoglobulin gamma 4 may have or may include the protein sequence of SEQ ID NO: 5 (EMBL/GenBank accession number AY445518). The heavy chain of the horse (Equus caballus) antibody immunoglobulin delta may have or may include the protein sequence of SEQ ID NO: 6 (EMBL/GenBank accession number AY631942). The heavy chain of the dog (Canis familiaris) antibody immunoglobulin A may have or may include the protein sequence of SEQ ID NO: 7 (GenBank accession number L36871). The heavy chain of the dog (Canis familiaris) antibody immunoglobulin E may have or may include the protein sequence of SEQ ID NO: 8 (GenBank accession number L36872). The heavy chain of the cat (Fells catus) antibody immunoglobulin G2 may have or may include the protein sequence of SEQ ID NO: 9 (DDBJ/EMBL/GenBank accession number KF811175).

Animals of the camelid family, such as camels (Camelus dromedarius and Camelus bactrianus), llamas (Lama glama, Lama pacos and Lama vicugna), alpacas (Vicugna pacos) and guanacos (Lama guanicoe), have a unique antibody that is not found in other mammals. In addition to conventional immunoglobulin G antibodies composed of heavy and light chain tetramers, camelids also have heavy chain immunoglobulin G antibodies that do not contain light chains and exist as heavy chain dimers. These antibodies are known as heavy chain antibodies, HCAbs, single-domain antibodies or sdAbs, and the variable domain of a camelid heavy chain antibody is known as the VHH. The camelid heavy chain antibodies lack the heavy chain CH1 domain and have a hinge region that is not found in other species. The variable region of the Arabian camel (Camelus dromedarius) single-domain antibody may have or may include the protein sequence of SEQ ID NO: 10 (GenBank accession number AJ245148). The variable region of the heavy chain of the Arabian camel (Camelus dromedarius) tetrameric immunoglobulin may have or may include the protein sequence of SEQ ID NO: 11 (GenBank accession number AJ245184).

In addition to camelids, heavy chain antibodies are also found in cartilaginous fishes, such as sharks, skates and rays. This type of antibody is known as an immunoglobulin new antigen receptor or IgNAR, and the variable domain of an IgNAR is known as the VNAR. The IgNAR exists as two identical heavy chain dimers composed of one variable domain and five constant domains each. Like camelids, there is no light chain.

The protein sequences of additional non-human species may be readily found in online databases, such as the International ImMunoGeneTics Information System (www.imgt.org), the European Bioinformatics Institute (www.ebi.ac.uk), the DNA Databank of Japan (ddbj.nig.ac.jp/arsa) or the National Center for Biotechnology Information (www.ncbi.nlm.nih.gov).

An anti-AGE antibody or a variant thereof may include a heavy chain having at least 90%, 91%, 92%, 93%, 94%, 95%, 96%, 97%, 98%, 99%, or 100% sequence identity to the amino acid sequence of SEQ ID NO: 1, SEQ ID NO: 16, SEQ ID NO: 17, SEQ ID NO: 29, SEQ ID NO: 31, SEQ ID NO: 33, SEQ ID NO: 47, SEQ ID NO: 48, SEQ ID NO: 49, SEQ ID NO: 50 or SEQ ID NO: 51, including post-translational modifications thereof. A heavy chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE.

An anti-AGE antibody or a variant thereof may include a heavy chain variable region having at least 90%, 91%, 92%, 93%, 94%, 95%, 96%, 97%, 98%, 99%, or 100% sequence identity to the amino acid sequence of SEQ ID NO: 2, SEQ ID NO: 20, SEQ ID NO: 23, SEQ ID NO: 24, SEQ ID NO: 25, SEQ ID NO: 41, SEQ ID NO: 42, SEQ ID NO: 43, SEQ ID NO: 52, SEQ ID NO: 53, SEQ ID NO: 54, SEQ ID NO: 55, or SEQ ID NO: 56, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain having at least 90%, 91%, 92%, 93%, 94%, 95%, 96%, 97%, 98%, 99%, or 100% sequence identity to the amino acid sequence of SEQ ID NO: 3, SEQ ID NO: 18, SEQ ID NO: 19, SEQ ID NO: 35, SEQ ID NO: 37, SEQ ID NO: 39, SEQ ID NO: 57, SEQ ID NO: 58, SEQ ID NO: 59, SEQ ID NO: 60 or SEQ ID NO: 61, including post-translational modifications thereof. A light chain having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

An anti-AGE antibody or a variant thereof may include a light chain variable region having at least 90%, 91%, 92%, 93%, 94%, 95%, 96%, 97%, 98%, 99%, or 100% sequence identity to the amino acid sequence of SEQ ID NO: 4, SEQ ID NO: 21, SEQ ID NO: 26, SEQ ID NO: 27, SEQ ID NO: 28, SEQ ID NO: 44, SEQ ID NO: 45, SEQ ID NO: 46, SEQ ID NO: 62, SEQ ID NO: 63, SEQ ID NO: 64, SEQ ID NO: 65 or SEQ ID NO: 66, including post-translational modifications thereof. A variable region having at least 90%, 91%, 92%, 93%, 94%, 95%, 96%, 97%, 98%, or 99% sequence identity may contain substitutions (e.g., conservative substitutions), insertions, or deletions relative to the reference sequence, but an anti-AGE antibody including that sequence retains the ability to bind to AGE. The substitutions, insertions, or deletions may occur in regions outside the variable region.

Alternatively, the antibody may have the complementarity determining regions of commercially available mouse anti-glycation end-product antibody raised against carboxymethyl lysine conjugated with keyhole limpet hemocyanin (CML-KLH), the carboxymethyl lysine MAb (Clone 318003) available from R&D Systems, Inc. (Minneapolis, Minn.; catalog no. MAB3247).

The antibody may have or may include constant regions which permit destruction of targeted cells by a subject's immune system.

Mixtures of antibodies that bind to more than one type AGE of AGE-modified proteins may also be used.

Bi-specific antibodies, which are anti-AGE antibodies directed to two different epitopes, may also be used. Such antibodies will have a variable region (or complementary determining region) from those of one anti-AGE antibody, and a variable region (or complementary determining region) from a different antibody.

Antibody fragments may be used in place of whole antibodies. For example, immunoglobulin G may be broken down into smaller fragments by digestion with enzymes. Papain digestion cleaves the N-terminal side of inter-heavy chain disulfide bridges to produce Fab fragments. Fab fragments include the light chain and one of the two N-terminal domains of the heavy chain (also known as the Fd fragment). Pepsin digestion cleaves the C-terminal side of the inter-heavy chain disulfide bridges to produce F(ab′)2 fragments. F(ab′)2 fragments include both light chains and the two N-terminal domains linked by disulfide bridges. Pepsin digestion may also form the Fv (fragment variable) and Fc (fragment crystallizable) fragments. The Fv fragment contains the two N-terminal variable domains. The Fc fragment contains the domains which interact with immunoglobulin receptors on cells and with the initial elements of the complement cascade. Pepsin may also cleave immunoglobulin G before the third constant domain of the heavy chain (C_{H}3) to produce a large fragment F(abc) and a small fragment pFc′. Antibody fragments may alternatively be produced recombinantly. Preferably, such antibody fragments are conjugated to an agent that causes the destruction of AGE-modified cells.

If additional antibodies are desired, they can be produced using well-known methods. For example, polyclonal antibodies (pAbs) can be raised in a mammalian host by one or more injections of an immunogen, and if desired, an adjuvant. Typically, the immunogen (and adjuvant) is injected in a mammal by a subcutaneous or intraperitoneal injection. The immunogen may be an AGE-modified protein of a cell, such as AGE-antithrombin III, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-RNAse, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Examples of adjuvants include Freund's complete, monophosphoryl Lipid A synthetic-trehalose dicorynomycolate, aluminum hydroxide (alum), heat shock proteins HSP 70 or HSP96, squalene emulsion containing monophosphoryl lipid A, a2-macroglobulin and surface active substances, including oil emulsions, pleuronic polyols, polyanions and dinitrophenol. To improve the immune response, an immunogen may be conjugated to a polypeptide that is immunogenic in the host, such as keyhole limpet hemocyanin (KLH), serum albumin, bovine thyroglobulin, cholera toxin, labile enterotoxin, silica particles or soybean trypsin inhibitor. A preferred immunogen conjugate is AGE-KLH. Alternatively, pAbs may be made in chickens, producing IgY molecules.

Monoclonal antibodies (mAbs) may also be made by immunizing a host or lymphocytes from a host, harvesting the mAb-secreting (or potentially secreting) lymphocytes, fusing those lymphocytes to immortalized cells (for example, myeloma cells), and selecting those cells that secrete the desired mAb. Other techniques may be used, such as the EBV-hybridoma technique. Non-human antibodies may be made less immunogenic to humans by engineering the antibodies to contain a combination of non-human and human antibody components. A chimeric antibody may be produced by combining the variable region of a non-human antibody with a human constant region. A humanized antibody may be produced by replacing the complementarity determining regions (CDRs) of a human antibody with those of a non-human antibody. Similarly, antibodies may be made less immunogenic to other species by being substantially “ized” to a given animal, such as cat, dog, horse, camel or alpaca, cattle, sheep, pig, or goat, at the amino acid level. If desired, the mAbs may be purified from the culture medium or ascites fluid by conventional procedures, such as protein A-sepharose, hydroxyapatite chromatography, gel electrophoresis, dialysis, ammonium sulfate precipitation or affinity chromatography. Additionally, human monoclonal antibodies can be generated by immunization of transgenic mice containing a third copy IgG human trans-loci and silenced endogenous mouse Ig loci or using human-transgenic mice. Production of humanized monoclonal antibodies and fragments thereof can also be generated through phage display technologies.

A “pharmaceutically acceptable carrier” includes any and all solvents, dispersion media, coatings, antibacterial and antifungal agents, isotonic and absorption delaying agents, and the like, compatible with pharmaceutical administration. Preferred examples of such carriers or diluents include water, saline, Ringer's solutions and dextrose solution. Supplementary active compounds can also be incorporated into the compositions. Solutions and suspensions used for parenteral administration can include a sterile diluent, such as water for injection, saline solution, polyethylene glycols, glycerin, propylene glycol or other synthetic solvents; antibacterial agents such as benzyl alcohol or methyl parabens; antioxidants such as ascorbic acid or sodium bisulfite; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The antibodies may be administered by injection, such as by intravenous injection or locally, such as by intra-articular injection into a joint. Pharmaceutical compositions suitable for injection include sterile aqueous solutions or dispersions for the extemporaneous preparation of sterile injectable solutions or dispersion. Various excipients may be included in pharmaceutical compositions of antibodies suitable for injection. Suitable carriers include physiological saline, bacteriostatic water, CREMOPHOR EL® (BASF; Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. Various antibacterial and anti-fungal agents, for example, parabens, chlorobutanol, phenol, ascorbic acid, and thimerosal, can contain microorganism contamination. Isotonic agents such as sugars, polyalcohols, such as mannitol, sorbitol, and sodium chloride can be included in the composition. Compositions that can delay absorption include agents such as aluminum monostearate and gelatin. Sterile injectable solutions can be prepared by incorporating antibodies, and optionally other therapeutic components, in the required amount in an appropriate solvent with one or a combination of ingredients as required, followed by sterilization. Methods of preparation of sterile solids for the preparation of sterile injectable solutions include vacuum drying and freeze-drying to yield a solid.

For administration by inhalation, the antibodies may be delivered as an aerosol spray from a nebulizer or a pressurized container that contains a suitable propellant, for example, a gas such as carbon dioxide. Antibodies may also be delivered via inhalation as a dry powder, for example using the iSPERSE™ inhaled drug delivery platform (PULMATRIX, Lexington, Mass.). The use of anti-AGE antibodies which are chicken antibodies (IgY) may be non-immunogenic in a variety of animals, including humans, when administered by inhalation.

An appropriate dosage level of each type of antibody will generally be about 0.01 to 500 mg per kg patient body weight. Preferably, the dosage level will be about 0.1 to about 250 mg/kg; more preferably about 0.5 to about 100 mg/kg. A suitable dosage level may be about 0.01 to 250 mg/kg, about 0.05 to 100 mg/kg, or about 0.1 to 50 mg/kg. Within this range the dosage may be 0.05 to 0.5, 0.5 to 5 or 5 to 50 mg/kg. Although each type of antibody may be administered on a regimen of 1 to 4 times per day, such as once or twice per day, antibodies typically have a long half-life in vivo. Accordingly, each type of antibody may be administered once a day, once a week, once every two or three weeks, once a month, or once every 60 to 90 days.

A subject that receives administration of an anti-AGE antibody may be tested to determine if the administration has been effective to treat a chronic effect of radiation or chemical exposure, such as symptoms which premature aging. For example, a subject may be considered to have received an effective antibody treatment if he or she demonstrates a reduction in one or more symptoms which mimic premature aging between subsequent measurements or over time. Alternatively, the concentration and/or number of senescent cells may be measured over time. Administration of antibody and subsequent testing may be repeated until the desired therapeutic result is achieved.

Unit dosage forms can be created to facilitate administration and dosage uniformity. Unit dosage form refers to physically discrete units suited as single dosages for the subject to be treated, containing a therapeutically effective quantity of one or more types of antibodies in association with the required pharmaceutical carrier. Preferably, the unit dosage form is in a sealed container and is sterile.

Vaccines against AGE-modified proteins or peptides contain an AGE antigen, an adjuvant, optional preservatives and optional excipients. Examples of AGE antigens include AGE-modified proteins or peptides such as AGE-antithrombin Ill, AGE-calmodulin, AGE-insulin, AGE-ceruloplasmin, AGE-collagen, AGE-cathepsin B, AGE-albumin such as AGE-bovine serum albumin (AGE-BSA), AGE-human serum albumin and ovalbumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-RNAse, AGE-hemoglobin such as AGE-human hemoglobin, AGE-low density lipoprotein (AGE-LDL) and AGE-collagen IV. AGE-modified cells, such as AGE-modified erythrocytes, whole, lysed, or partially digested, may also be used as AGE antigens. Suitable AGE antigens also include proteins or peptides that exhibit AGE modifications (also referred to as AGE epitopes or AGE moieties) such as carboxymethyllysine (CML), carboxyethyllysine (CEL), pentosidine, pyrraline, FFI, AFGP and ALI. The AGE antigen may be an AGE-protein conjugate, such as AGE conjugated to keyhole limpet hemocyanin (AGE-KLH). Further details of some of these AGE-modified proteins or peptides and their preparation are described in Bucala.

Particularly preferred AGE antigens include proteins or peptides that exhibit a carboxymethyllysine or carboxyethyllysine AGE modification. Carboxymethyllysine (also known as N(epsilon)-(carboxymethyl)lysine, N(6)-carboxymethyllysine, or 2-Amino-6-(carboxymethylamino)hexanoic acid) and carboxyethyllysine (also known as N-epsilon-(carboxyethyl)lysine) are found on proteins or peptides and lipids as a result of oxidative stress and chemical glycation, and have been correlated with juvenile genetic disorders. CML- and CEL-modified proteins or peptides are recognized by the receptor RAGE which is expressed on a variety of cells. CML and CEL have been well-studied and CML- and CEL-related products are commercially available. For example, Cell Biolabs, Inc. sells CML-BSA antigens, CML polyclonal antibodies, CML immunoblot kits, and CML competitive ELISA kits (www.cellbiolabs.com/cml-assays) as well as CEL-BSA antigens and CEL competitive ELISA kits (www.cellbiolabs.com/cel-n-epsilon-carboxyethyl-lysine-assays-and-reagents).

AGE antigens may be conjugated to carrier proteins to enhance antibody production in a subject. Antigens that are not sufficiently immunogenic alone may require a suitable carrier protein to stimulate a response from the immune system. Examples of suitable carrier proteins include keyhole limpet hemocyanin (KLH), serum albumin, bovine thyroglobulin, cholera toxin, labile enterotoxin, silica particles and soybean trypsin inhibitor. Preferably, the carrier protein is KLH (AGE-KLH). KLH has been extensively studied and has been identified as an effective carrier protein in experimental cancer vaccines. Preferred AGE antigen-carrier protein conjugates include CML-KLH and CEL-KLH.

The administration of an AGE antigen allows the immune system to develop immunity to the antigen. Immunity is a long-term immune response, either cellular or humoral. A cellular immune response is activated when an antigen is presented, preferably with a co-stimulator to a T-cell which causes it to differentiate and produce cytokines. The cells involved in the generation of the cellular immune response are two classes of T-helper (Th) cells, Th1 and Th2. Th1 cells stimulate B cells to produce predominantly antibodies of the IgG2A isotype, which activates the complement cascade and binds the Fc receptors of macrophages, while Th2 cells stimulate B cells to produce IgG1 isotype antibodies in mice, IgG4 isotype antibodies in humans, and IgE isotype antibodies. The human body also contains “professional” antigen-presenting cells such as dendritic cells, macrophages, and B cells.

A humoral immune response is triggered when a B cell selectively binds to an antigen and begins to proliferate, leading to the production of a clonal population of cells that produce antibodies that specifically recognize that antigen and which may differentiate into antibody-secreting cells, referred to as plasma-cells or memory-B cells. Antibodies are molecules produced by B-cells that bind a specific antigen. The antigen-antibody complex triggers several responses, either cell-mediated, for example by natural killers (NK) or macrophages, or serum-mediated, for example by activating the complement system, a complex of several serum proteins that act sequentially in a cascade that result in the lysis of the target cell.

Immunological adjuvants (also referred to simply as “adjuvants”) are the component(s) of a vaccine which augment the immune response to the immunogenic agent. Adjuvants function by attracting macrophages to the immunogenic agent and then presenting the agent to the regional lymph nodes to initiate an effective antigenic response. Adjuvants may also act as carriers themselves for the immunogenic agent. Adjuvants may induce an inflammatory response, which may play an important role in initiating the immune response.

Adjuvants include mineral compounds such as aluminum salts, oil emulsions, bacterial products, liposomes, immunostimulating complexes and squalene. Aluminum compounds are the most widely used adjuvants in human and veterinary vaccines. These aluminum compounds include aluminum salts such as aluminum phosphate (AlPO₄) and aluminum hydroxide (Al(OH)₃) compounds, typically in the form of gels, and are generically referred to in the field of vaccine immunological adjuvants as “alum.” Aluminum hydroxide is a poorly crystalline aluminum oxyhydroxide having the structure of the mineral boehmite. Aluminum phosphate is an amorphous aluminum hydroxyphosphate. Negatively charged species (for example, negatively charged antigens) can absorb onto aluminum hydroxide gels at neutral pH, whereas positively charged species (for example, positively charged antigens) can absorb onto aluminum phosphate gels at neutral pH. It is believed that these aluminum compounds provide a depot of antigen at the site of administration, thereby providing a gradual and continuous release of antigen to stimulate antibody production. Aluminum compounds tend to more effectively stimulate a cellular response mediated by Th2, rather than Th1 cells.

Emulsion adjuvants include water-in-oil emulsions (for example, Freund's adjuvants, such as killed mycobacteria in oil emulsion) and oil-in-water emulsions (for example, MF-59). Emulsion adjuvants include an immunogenic component, for example squalene (MF-59) or mannide oleate (Incomplete Freund's Adjuvants), which can induce an elevated humoral response, increased T cell proliferation, cytotoxic lymphocytes and cell-mediated immunity.

Liposomal or vesicular adjuvants (including paucilamellar lipid vesicles) have lipophilic bilayer domains and an aqueous milieu which can be used to encapsulate and transport a variety of materials, for example an antigen. Paucilamellar vesicles (for example, those described in U.S. Pat. No. 6,387,373) can be prepared by mixing, under high pressure or shear conditions, a lipid phase comprising a non-phospholipid material (for example, an amphiphile surfactant; see U.S. Pat. Nos. 4,217,344; 4,917,951; and 4,911,928), optionally a sterol, and any water-immiscible oily material to be encapsulated in the vesicles (for example, an oil such as squalene oil and an oil-soluble or oil-suspended antigen); and an aqueous phase such as water, saline, buffer or any other aqueous solution used to hydrate the lipids. Liposomal or vesicular adjuvants are believed to promote contact of the antigen with immune cells, for example by fusion of the vesicle to the immune cell membrane, and preferentially stimulate the Th1 sub-population of T-helper cells.

Other types of adjuvants include Mycobacterium bovis bacillus Calmette-Guérin (BCG), quill-saponin and unmethylated CpG dinucleotides (CpG motifs). Additional adjuvants are described in U.S. Patent Application Publication Pub. No. US 2010/0226932 (Sep. 9, 2010) and Jiang, Z-H. et al. “Synthetic vaccines: the role of adjuvants in immune targeting”, Current Medicinal Chemistry, Vol. 10(15), pp. 1423-39 (2003). Preferable adjuvants include Freund's complete adjuvant and Freund's incomplete adjuvant.

The vaccine may optionally include one or more preservatives, such as antioxidants, antibacterial and antimicrobial agents, as well as combinations thereof. Examples include benzethonium chloride, ethylenediamine-tetraacetic acid sodium (EDTA), thimerosal, phenol, 2-phenoxyethanol, formaldehyde and formalin; antibacterial agents such as amphotericin B, chlortetracycline, gentamicin, neomycin, polymyxin B and streptomycin; antimicrobial surfactants such as polyoxyethylene-9, 10-nonyl phenol (Triton N-101, octoxynol-9), sodium deoxycholate and polyoxyethylated octyl phenol (Triton X-100). The production and packaging of the vaccine may eliminate the need for a preservative. For example, a vaccine that has been sterilized and stored in a sealed container may not require a preservative.

Other components of vaccines include pharmaceutically acceptable excipients, such as stabilizers, thickening agents, toxin detoxifiers, diluents, pH adjusters, tonicity adjustors, surfactants, antifoaming agents, protein stabilizers, dyes and solvents. Examples of such excipients include hydrochloric acid, phosphate buffers, sodium acetate, sodium bicarbonate, sodium borate, sodium citrate, sodium hydroxide, potassium chloride, potassium chloride, sodium chloride, polydimethylsilozone, brilliant green, phenol red (phenolsulfon-phthalein), glycine, glycerin, sorbitol, histidine, monosodium glutamate, potassium glutamate, sucrose, urea, lactose, gelatin, sorbitol, polysorbate 20, polysorbate 80 and glutaraldehyde. A variety of these components of vaccines, as well as adjuvants, are described in www.cdc.gov/vaccines/pubs/pinkbook/downloads/appendices/B/excipient-table-2.pdf and Vogel, F. R. et al., “A compendium of vaccine adjuvants and excipients”, Pharmaceutical Biotechnology, Vol. 6, pp. 141-228 (1995).

The vaccine may contain from 1 μg to 100 mg of at least one AGE antigen, including 10, 20, 30, 40, 50, 60, 70, 80, 90, 100, 200, 400, 800 or 1000 μg, or 2, 3, 4, 5, 6, 7, 8, 9, 10, 20, 30, 40, 50, 60, 70, 80 or 90 mg. The amount used for a single injection corresponds to a unit dosage.

The vaccine may be provided in unit dosage form or in multidosage form, such as 2-100 or 2-10 doses. The unit dosages may be provided in a vial with a septum, or in a syringe with or without a needle. The vaccine may be administered intravenously, subdermally or intraperitoneally. Preferably, the vaccine is sterile.

The vaccine may be administered one or more times, such as 1 to 10 times, including 2, 3, 4, 5, 6, 7, 8 or 9 times, and may be administered over a period of time ranging from 1 week to 1 year, 2-10 weeks or 2-10 months. Furthermore, booster vaccinations may be desirable, over the course of 1 year to 20 years, including 2, 5, 10 and 15 years.

A subject that receives a vaccine for AGE-modified proteins or peptides of a cell may be tested to determine if he or she has developed an immunity to the AGE-modified proteins or peptides. Suitable tests may include blood tests for detecting the presence of an antibody, such as immunoassays or antibody titers. An immunity to AGE-modified proteins or peptides may also be determined by monitoring the concentration and/or number of senescent cells over time. In addition to testing for the development of an immunity to AGE-modified proteins or peptides, a subject may also be tested to determine if the vaccination has been effective to treat a chronic effect of radiation or chemical exposure, such as symptoms which mimic premature aging. For example, a subject may be considered to have received an effective vaccination if he or she demonstrates a reduction in one or more symptoms which mimic premature aging between subsequent measurements or over time, or by measuring the concentration and/or number of senescent cells. Vaccination and subsequent testing may be repeated until the desired therapeutic result is achieved.

The vaccination process may be designed to provide immunity against multiple AGE moieties. A single AGE antigen may induce the production of AGE antibodies which are capable of binding to multiple AGE moieties. Alternatively, the vaccine may contain multiple AGE antigens. In addition, a subject may receive multiple vaccines, where each vaccine contains a different AGE antigen.

Any mammal may be treated by the methods herein described. Humans are a preferred mammal for treatment. Other mammals that may be treated include mice, rats, goats, sheep, pigs, cows, horses and companion animals, such as dogs or cats. Alternatively, any of the mammals or subjects identified above may be excluded from the patient population in need of treatment for pain associated with inflammation.

A subject may be identified as in need of treatment based on the presence of one or more chronic effects of radiation or chemical exposure, such as symptoms which mimic premature aging. Symptoms which mimic premature aging include the development of gray hair, wrinkles, frailty, cataracts, arteriosclerosis, atherosclerosis, Alzheimer's disease, Parkinson's disease, sarcopenia, loss of adipose tissue, lordokyphosis, cancer, premature menopause, cardiovascular disease, dementia, Type II diabetes, endocrinopathies, cardiac dysfunction, osteoporosis, osteoarthritis, pulmonary fibrosis, kidney and liver disease, metabolic disorders, lipodystrophy, hearing loss, vision loss and memory loss. A subject may also be identified as in need of treatment based on a diagnosis with one or more progeroid syndromes, including Hutchinson-Gilford progeria syndrome (also known as progeria), Werner syndrome, Bloom syndrome, Rothmund-Thomson syndrome, Cockayne syndrome, xeroderma pigmentosum, trichothiodystrophy, combined xeroderma pigmentosum-Cockayne syndrome and restrictive dermopathy. In addition, subjects may be identified as in need of treatment based on the presence of a pathological condition associated with inflammation or AGEs such as, for example, metastatic cancer, retinopathy, nephropathy, stroke, endothelial cell dysfunction or neurodegenerative disorders.

A subject also may be identified as in need of treatment based on a known or anticipated exposure to radiation or chemicals. For example, a subject may be identified as in need of treatment after exposure to chemical weapons such as chlorine gas, phosgene gas, mustard gas, a G-series nerve agent, a V-series nerve agent, Novichok agents, carbamates or insecticides, or after exposure to poisons such as dioxin, lead or cadmium. Similarly, a subject who has received or is about to begin receiving chemotherapy or HAART may be identified as in need of treatment. Examples of commonly used chemotherapy agents include vinorelbine (NAVELBINE®), mitomycin (MITOSOL®), camptothecin, cyclophosphamide (CYTOXAN®), methotrexate (TREXALL®), tamoxifen citrate (NOLVADEX®, SOLTAMOX®), 5-fluorouracil (ADRUCIL®), irinotecan (ONIVYDE®), doxorubicin (DOXIL®), flutamide, paclitaxel (TAXOL®, ABRAXANE®), docetaxel (DOCEFREZ®, TAXOTERE®), vinblastine, imatinib mesylate (GLEEVEC®), anthracycline, letrozole (FEMARA®), arsenic trioxide (TRISENOX®), anastrozole (ARIMIDEX®), triptorelin pamoate (TRELSTAR®), ozogamicin, irinotecan hydrochloride (CAMPTOSAR®), BCG live (THERACYS®), leuprolide acetate implant (VIADUR®), bexarotene (TARGRETIN®), exemestane (AROMASIN®), topotecan hydrochloride (HYCAMTIN®), gemcitabine HCL (GEMZAR®), daunorubicin hydrochloride, toremifene citrate (FARESTON®), carboplatin (PARAPLATIN®), cisplatin (PLATINOL®), oxaliplatin (ELOTAXIN®) and any other platinum-containing oncology drug, trastuzumab (HERCEPTIN®), lapatinib (TYKERB®), gefitinib (IRESSA®), cetuximab (ERBITUX®), panitumumab (VECTIBIX®), temsirolimus (TORISEL®), everolimus (AFINITOR®), vandetanib (CAPRELSA®), vemurafenib (ZELBORAF®), crizotinib (XALKORI®), vorinostat (ZOLINZA®), bevacizumab (AVASTIN®), radiation therapy, hyperthermia, gene therapy and photodynamic therapy. A chemotherapy or HAART treatment regimen may combine administration of a chemotherapeutic agent or antiretroviral agent with administration of an anti-AGE antibody or vaccination against AGE-modified proteins or AGE-modified peptides.

The one-letter amino acid sequence that corresponds to SEQ ID NO: 1 is shown below:

```
                10 20 30 40
MNLLLILTFV AAAVAQVQLL QPGAELVKPG ASVKLACKAS
                50 60 70 80
GYLFTTYWMH WLKQRPGQGL EWIGEISPTN GRAYYNARFK
                90 100 110 120
SEATLTVDKS SNTAYMQLSS LTSEASAVYY CARAYGNYEF
              130 140 150 160
AYWGQGTLVT VSVASTKGPS VFPLAPSSKS TSGGTAALGC
              170 180 190 200
LVKDYFPEPV TVSWNSGALT SGVHTFPAVL QSSGLYSLSS
              210 220 230 240
VVTVPSSSLG TQTYICNVNH KPSNTKVDKK VEPKSCDKTH
              250 260 270 280
TCPPCPAPEL LGGPSVFLFP PKPKDTLMIS RTPEVTCVVV
              290 300 310 320
DVSHEDPEVK FNWYVDGVEV HNAKTKPREE QYNSTYRVVS
              330 340 350 360
VLTVLHQDWL NGKEYKCKVS NKALPAPIEK TISKAKGQPR
              370 380 390 400
EPQVYTLPPS REEMTKNQVS LTCLVKGFYP SDIAVEWESN
              410 420 430 440
GQPENNYKTT PPVLDSDGSF FLYSKLTVDK SRWQQGNVFS
              450 460
CSVMHEALHN HYTQKSLSLS PGK
```

Positions 16-133 of the above amino acid sequence correspond to SEQ ID NO: 2. Positions 46-50 of the above amino acid sequence correspond to SEQ ID NO: 41. Positions 65-81 of the above amino acid sequence correspond to SEQ ID NO: 42. Positions 114-122 of the above amino acid sequence correspond to SEQ ID NO: 43.

The one-letter amino acid sequence that corresponds to SEQ ID NO: 3 is shown below:

```
                10 20 30 40
MNLLLILTFV AAAVADVVMT QTPLSLPVSL GDQASISCRS
                50 60 70 80
RQSLVNSNGN TFLQWYLQKP GQSPKLLIYK VSLRFSGVPD
                90 100 110 120
RFSGSGSGTD FTLKISRVEA EDLGLYFCSQ STHVPPTFGG
              130 140 150 160
GTKLEIKRTV AAPSVFIFPP SDEQLKSGTA SVVCLLNNFY
              170 180 190 200
PREAKVQWKV DNALQSGNSQ ESVTEQDSKD STYSLSSTLT
              210 220 230
LSKADYEKHK VYACEVTHQG LSSPVTKSFN RGEC
```

Positions 16-128 of the above amino acid sequence correspond to SEQ ID NO: 4. Optionally, the arginine (Arg or R) residue at position 128 of SEQ ID NO: 4 may be omitted. Positions 39-54 of the above amino acid sequence correspond to SEQ ID NO: 44. Positions 70-76 of the above amino acid sequence correspond to SEQ ID NO: 45. Positions 109-117 of the above amino acid sequence correspond to SEQ ID NO: 46.

The DNA sequence that corresponds to SEQ ID NO: 12 is shown below:

ATGGACCCCAAGGGCAGCCTGAGCTGGAGAATCCTGCTGTTCCTGAGCCT
GGCCTTCGAGCTGAGCTACGGCCAGGTGCAGCTGCTGCAGCCAGGTGCCG
AGCTCGTGAAACCTGGCGCCTCTGTGAAGCTGGCCTGCAAGGCTTCCGGC
TACCTGTTCACCACCTACTGGATGCACTGGCTGAAGCAGAGGCCAGGCCA
GGGCCTGGAATGGATCGGCGAGATCTCCCCCACCAACGGCAGAGCCTACT
ACAACGCCCGGTTCAAGTCCGAGGCCACCCTGACCGTGGACAAGTCCTCC
AACACCGCCTACATGCAGCTGTCCTCCCTGACCTCTGAGGCCTCCGCCGT
GTACTACTGCGCCAGAGCTTACGGCAACTACGAGTTCGCCTACTGGGGCC
AGGGCACCCTCGTGACAGTGTCTGTGGCTAAGACCACCCCTCCCTCCGTG
TACCCTCTGGCTCCTGGCTGTGGCGACACCACCGGATCCTCTGTGACCCT
GGGCTGCCTCGTGAAGGGCTACTTCCCTGAGTCCGTGACCGTGACCTGGA
ACTCCGGCTCCCTGTCCTCCTCCGTGCACACCTTTCCAGCCCTGCTGCAG
TCCGGCCTGTACACCATGTCCTCCAGCGTGACAGTGCCCTCCTCCACCTG
GCCTTCCCAGACCGTGACATGCTCTGTGGCCCACCCTGCCTCTTCCACCA
CCGTGGACAAGAAGCTGGAACCCTCCGGCCCCATCTCCACCATCAACCCT
TGCCCTCCCTGCAAAGAATGCCACAAGTGCCCTGCCCCCAACCTGGAAGG
CGGCCCTTCCGTGTTCATCTTCCCACCCAACATCAAGGACGTGCTGATGA
TCTCCCTGACCCCCAAAGTGACCTGCGTGGTGGTGGACGTGTCCGAGGAC
GACCCTGACGTGCAGATCAGTTGGTTCGTGAACAACGTGGAAGTGCACAC
CGCCCAGACCCAGACACACAGAGAGGACTACAACAGCACCATCAGAGTGG
TGTCTACCCTGCCCATCCAGCACCAGGACTGGATGTCCGGCAAAGAATTC
AAGTGCAAAGTGAACAACAAGGACCTGCCCAGCCCCATCGAGCGGACCAT
CTCCAAGATCAAGGGCCTCGTGCGGGCTCCCCAGGTGTACATTCTGCCTC
CACCAGCCGAGCAGCTGTCCCGGAAGGATGTGTCTCTGACATGTCTGGTC
GTGGGCTTCAACCCCGGCGACATCTCCGTGGAATGGACCTCCAACGGCCA
CACCGAGGAAAACTACAAGGACACCGCCCCTGTGCTGGACTCCGACGGCT
CCTACTTCATCTACTCCAAGCTGAACATGAAGACCTCCAAGTGGGAAAAG
ACCGACTCCTTCTCCTGCAACGTGCGGCACGAGGGCCTGAAGAACTACTA
CCTGAAGAAAACCATCTCCCGGTCCCCCGGCTAG

The DNA sequence that corresponds to SEQ ID NO: 13 is shown below:

ATGGACCCCAAGGGCAGCCTGAGCTGGAGAATCCTGCTGTTCCTGAGCCT
GGCCTTCGAGCTGAGCTACGGCCAGGTGCAGCTGCTGCAGCCAGGTGCCG
AGCTCGTGAAACCTGGCGCCTCTGTGAAGCTGGCCTGCAAGGCTTCCGGC
TACCTGTTCACCACCTACTGGATGCACTGGCTGAAGCAGAGGCCAGGCCA
GGGCCTGGAATGGATCGGCGAGATCTCCCCCACCAACGGCAGAGCCTACT
ACAACGCCCGGTTCAAGTCCGAGGCCACCCTGACCGTGGACAAGTCCTCC
AACACCGCCTACATGCAGCTGTCCTCCCTGACCTCTGAGGCCTCCGCCGT
GTACTACTGCGCCAGAGCTTACGGCAACTACGAGTTCGCCTACTGGGGCC
AGGGCACCCTCGTGACAGTGTCTGTGGCTAGCACCAAGGGCCCCAGCGTG
TTCCCTCTGGCCCCCAGCAGCAAGAGCACCAGCGGCGGAACCGCCGCCCT
GGGCTGCCTGGTGAAGGACTACTTCCCCGAGCCCGTGACCGTGTCCTGGA
ACAGCGGCGCTCTGACCAGCGGAGTGCACACCTTCCCTGCCGTGCTGCAG
AGCAGCGGCCTGTACTCCCTGAGCAGCGTGGTGACCGTGCCCAGCAGCAG
CCTGGGCACCCAGACCTACATCTGCAACGTGAACCACAAGCCCTCCAACA
CCAAGGTGGACAAGAAGGTGGAGCCTAAGAGCTGCGACAAGACCCACACC
TGCCCTCCCTGCCCCGCCCCCGAGCTGCTGGGCGGACCCAGCGTGTTCCT
GTTCCCTCCCAAGCCCAAGGACACCCTGATGATCAGCCGCACCCCCGAGG
TGACCTGCGTGGTGGTGGACGTGAGCCACGAGGACCCCGAGGTGAAGTTC
AACTGGTACGTGGACGGCGTGGAGGTGCACAACGCCAAGACCAAGCCTCG
GGAGGAGCAGTACAACTCCACCTACCGCGTGGTGAGCGTGCTGACCGTGC
TGCACCAGGACTGGCTGAACGGCAAGGAGTACAAGTGCAAGGTGAGCAAC
AAGGCCCTGCCCGCTCCCATCGAGAAGACCATCAGCAAGGCCAAGGGCCA
GCCCCGGGAGCCTCAGGTGTACACCCTGCCCCCCAGCCGCGACGAGCTGA
CCAAGAACCAGGTGAGCCTGACCTGCCTGGTGAAGGGCTTCTACCCCTCC
GACATCGCCGTGGAGTGGGAGAGCAACGGCCAGCCTGAGAACAACTACAA
GACCACCCCTCCCGTGCTGGACAGCGACGGCAGCTTCTTCCTGTACAGCA
AGCTGACCGTGGACAAGTCCCGGTGGCAGCAGGGCAACGTGTTCAGCTGC
AGCGTGATGCACGAGGCCCTGCACAACCACTACACCCAGAAGAGCCTGAG
CCTGAGCCCCGGATAG

The DNA sequence that corresponds to SEQ ID NO: 14 is shown below:

ATGGAGACCGACACCCTGCTGCTCTGGGTGCTGCTGCTCTGGGTGCCCGG
CTCCACCGGAGACGTCGTGATGACCCAGACCCCTCTGTCCCTGCCTGTGT
CTCTGGGCGACCAGGCCTCCATCTCCTGCCGGTCTAGACAGTCCCTCGTG
AACTCCAACGGCAACACCTTCCTGCAGTGGTATCTGCAGAAGCCCGGCCA
GTCCCCCAAGCTGCTGATCTACAAGGTGTCCCTGCGGTTCTCCGGCGTGC
CCGACAGATTTTCCGGCTCTGGCTCTGGCACCGACTTCACCCTGAAGATC
TCCCGGGTGGAAGCCGAGGACCTGGGCCTGTACTTCTGCAGCCAGTCCAC
CCACGTGCCCCCTACATTTGGCGGAGGCACCAAGCTGGAAATCAAACGGG
CAGATGCTGCACCAACTGTATCCATCTTCCCACCATCCAGTGAGCAGTTA
ACATCTGGAGGTGCCTCAGTCGTGTGCTTCTTGAACAACTTCTACCCCAA
AGACATCAATGTCAAGTGGAAGATTGATGGCAGTGAACGACAAAATGGCG
TCCTGAACAGTTGGACTGATCAGGACAGCAAAGACAGCACCTACAGCATG
AGCAGCACCCTCACGTTGACCAAGGACGAGTATGAACGACATAACAGCTA
TACCTGTGAGGCCACTCACAAGACATCAACTTCACCCATTGTCAAGAGCT
TCAACAGGAATGAGTGTTGA

The DNA sequence that corresponds to SEQ ID NO: 15 is shown below:

ATGGAGACCGACACCCTGCTGCTCTGGGTGCTGCTGCTCTGGGTGCCCGG
CTCCACCGGAGACGTCGTGATGACCCAGACCCCTCTGTCCCTGCCTGTGT
CTCTGGGCGACCAGGCCTCCATCTCCTGCCGGTCTAGACAGTCCCTCGTG
AACTCCAACGGCAACACCTTCCTGCAGTGGTATCTGCAGAAGCCCGGCCA
GTCCCCCAAGCTGCTGATCTACAAGGTGTCCCTGCGGTMTCCGGCGTGCC
CGACAGATTTTCCGGCTCTGGCTCTGGCACCGACTTCACCCTGAAGATCT
CCCGGGTGGAAGCCGAGGACCTGGGCCTGTACTTCTGCAGCCAGTCCACC
CACGTGCCCCCTACATTTGGCGGAGGCACCAAGCTGGAAATCAAGCGGAC
CGTGGCCGCCCCCAGCGTGTTCATCTTCCCTCCCAGCGACGAGCAGCTGA
AGTCTGGCACCGCCAGCGTGGTGTGCCTGCTGAACAACTTCTACCCCCGC
GAGGCCAAGGTGCAGTGGAAGGTGGACAACGCCCTGCAGAGCGGCAACAG
CCAGGAGAGCGTGACCGAGCAGGACTCCAAGGACAGCACCTACAGCCTGA
GCAGCACCCTGACCCTGAGCAAGGCCGACTACGAGAAGCACAAGGTGTAC
GCCTGCGAGGTGACCCACCAGGGACTGTCTAGCCCCGTGACCAAGAGCTT
CAACCGGGGCGAGTGCTAA

The one-letter amino acid sequence that corresponds to SEQ ID NO: 16 is shown below:

MDPKGSLSWRILLFLSLAFELSYGQVQLLQPGAELVKPGASVKLACKASG
YLFTTYWMHWLKQRPGQGLEWIGEISPTNGRAYYNARFKSEATLTVDKSS
NTAYMQLSSLTSEASAVYYCARAYGNYEFAYWGQGTLVTVSVAKTTPPSV
YPLAPGCGDTTGSSVTLGCLVKGYFPESVNTWNSGSLSSSVHTFPALLQS
GLYTMSSSVTVPSSTWPSQTVTCSVAHPASSTTVDKKLEPSGPISTINPC
PPCKECHKCPAPNLEGGPSVFIFPPNIKDVLMISLTPKVTCVVVDVSEDD
PDVQISWFVNNVEVHTAQTQTHREDYNSTIRVVSTLPIQHQDWMSGKEFK
CKVNNKDLPSPIERTISKIKGLVRAPQVYILPPPAEQLSRKDVSLTCLVV
GFNPGDISVEWTSNGHTEENYKDTAPVLDSDGSYFIYSKLNMKTSKWEKT
DSFSCNVRHEGLKNYYLKKTISRSPG*

The alanine residue at position 123 of the above amino acid sequence may optionally be replaced with a serine residue. The tyrosine residue at position 124 of the above amino acid sequence may optionally be replaced with a phenylalanine residue. Positions 25-142 of the above amino acid sequence correspond to SEQ ID NO: 20. SEQ ID NO: 20 may optionally include the substitutions at positions 123 and 124. SEQ ID NO: 20 may optionally contain one additional lysine residue after the terminal valine residue.

The one-letter amino acid sequence that corresponds to SEQ ID NO: 17 is shown below:

MDPKGSLSWRILLFLSLAFELSYGQVQLLQPGAELVKPGASVKLACKASG
YLFTTYWMHWLKQRPGQGLEWIGEISPTNGRAYYNARFKSEATLIVDKSS
NTAYMQLSSLTSEASAVYYCARAYGNYEFAYWGQGTLVTVSVASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPG*

The one-letter amino acid sequence that corresponds to SEQ ID NO: 18 is shown below:

METDTLLLWVLLLWVPGSTGDVVMTQTPLSLPVSLGDQASISCRSRQSLV
NSNGNTFLQWYLQKPGQSPKLLIYKVSLRFSGVPDRFSGSGSGTDFTLKI
SRVEAEDLGLYFCSQSTHVPPTFGGGTKLEIKRADAAPTVSIFPPSSEQL
TSGGASVVCFLNNFYPKDINVKWKIDGSERQNGVLNSWTDQDSKDSTYSM
SSTLTLTKDEYERHNSYTCEATHKTSTSPIVKSFNRNEC*

Positions 21-132 of the above amino acid sequence correspond to SEQ ID NO: 21.

The one-letter amino acid sequence that corresponds to SEQ ID NO: 19 is shown below:

METDTLLLWVLLLWVPGSTGDVVMTQTPLSLPVSLGDQASISCRSRQSLV
NSNGNTFLQWYLQKPGQSPKLLIYKVSLRFSGVPDRFSGSGSGTDFTLKI
SRVEAEDLGLYFCSQSTHVPPTFGGGTKLEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC*

The one-letter amino acid sequence that corresponds to SEQ ID NO: 22 is shown below:

```
                10 20 30 40
ASTKGPSVFP LAPCSRSTSE STAALGCLVK DYFPEPVTVS
                50 60 70 80
WNSGALTSGV HTFPAVLQSS GLYSLSSVVT VPSSNFGTQT
                90 100 110 120
YTCNVDHKPS NTKVDKTVER KCCVECPPCP APPVAGPSVF
              130 140 150 160
LFPPKPKDTL MISRTPEVTC VVVDVSHEDP EVQFNWYVDG
              170 180 190 200
VEVHNAKTKP REEQFNSTFR VVSVLTVVHQ DWLNGKEYKC
              210 220 230 240
KVSNKGLPAP IEKTISKTKG QPREPQVYTL PPSREEMTKN
              250 260 270 280
QVSLTCLVKG FYPSDISVEW ESNGQPENNY KTTPPMLDSD
              290 300 310 320
GSFFLYSKLT VDKSRWQQGN VFSCSVMHEA LHNHYTQKSL
SLSPGK
```

The one-letter amino acid sequence that corresponds to SEQ ID NO: 23 is SYTMGVS.

The one-letter amino acid sequence that corresponds to SEQ ID NO: 24 is TISSGGGSTYYPDSVKG.

The one-letter amino acid sequence that corresponds to SEQ ID NO: 25 is QGGWLPPFAX, where X may be any naturally occurring amino acid.

The one-letter amino acid sequence that corresponds to SEQ ID NO: 26 is RASKSVSTSSRGYSYMH.

The one-letter amino acid sequence that corresponds to SEQ ID NO: 27 is LVSNLES.

The one-letter amino acid sequence that corresponds to SEQ ID NO: 28 is QHIRELTRS.

The one-letter amino acid sequence that corresponds to SEQ ID NO: 29 is

MDPKGSLSWRILLFLSLAFELSYGQVQLVQSGAEVKKPGASVKVSCKASG
YLFTTYWMHWVRQAPGQGLEWMGEISPTNGRAYYNQKFQGRVTMTVIDKS
TNTVYMELSSLRSEDTAVYYCARAYGNYFAYWGQGTLVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSRDELKNQVSLTCLVKGFYPSDI
AVEWESNGQPENNYKTTPPVLDSDGSFFLYSKLTVDKSRWQQGNVFSCSV
MHEALHNHYTQKSLSLSPG.

The DNA sequence that corresponds to SEQ ID NO: 30 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACAGAAATTCCAGGGCAGAGTGACCATGACCGTGGACAAGTCCACCAA
CACCGTGTACATGGAACTGTCCTCCCTGCGGAGCGAGGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCIGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 31 is

MDPKGSLSWRILLFLSLAFELSYGQVQLVQSGAEVKKPGASVKVSCKASG
YLFTTYWMHWVRQAPGQGLEWMGEISPTNGRAYYNAKFQGRVIMTVDKST
NTAYMELSSLRSEDTAVYYCARAYGNYFAYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTC
PPCPPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELKNQVSLTCLVKGFYPSDIA
VEWESNGQPENNYKTTPPVLDSDGSFFLYSKLTVDKSRWQQGNVFSCSVM
HEALHNHYTQKSLSLSPG.

The DNA sequence that corresponds to SEQ ID NO: 32 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACCAAAATTCCAGGGCAGAGTGACCATGACCGTGGACAAGTCCACCAA
CACCGCTTACATGGAACTGTCCTCCCTGCGGAGCGAGGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCTGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 33 is

MDPKGSLSWRILLFLSLAFELSYGQVQLVQSGAEVKKPGASVKVSCKASG
YLFTTYWMHWVRQAPGQGLEWMGEISPINGRAYYNAKFQGRVTMTVDKSI
NTAYMELSRLRSDDTAVYYCARAYGNYFAYWGQGTLVTVSSASTKGPSVF
PLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTC
PPCPPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELKNQVSLTCLVKGFYPSDIA
VEWESNGQPENNYKTTPPVLDSDGSFFLYSKLTVDKSRWQQGNVFSCSVM
HEALHNHYTQKSLSLSPG.

The DNA sequence that corresponds to SEQ ID NO: 34 is

ATGGACCCCAAGGGCAGCCTGAGCTGGAGAATCCTGCTGTTCCTGAGCCT
GGCCTTCGAGCTGAGCTACGGCCAGGTGCAGCTGGTGCAGTCTGGCGCCG
AAGTGAAGAAACCTGGCGCCTCCGTGAGGTGTCCTGCAAGGCTTCCGGCT
ACCTGTTCACCACCTACTGGATGCACTGGGTGCGACAGGCCCCTGGACAG
GGCCTGGAATGGATGGGCGAGATCTCCCCTACCAACGGCAGAGCCTACTA
CAACCAAAATTCCAGGGCAGAGTGACCATGACCGTGGACAAGTCCATCAA
CACCGCTTACATGGAACTGTCCAGACTGCGGAGCGATGACACCGCCGTGT
ACTACTGCGCTAGAGCCTACGGCAACTACGATTCGCCTACTGGGGCCAGG
GCACCCTCGTGACAGTGTCCTCTGCTAGCACCAAGGGCCCCAGCGTGTTC
CCTCTGGCCCCCAGCAGCAAGAGCACCAGCGGCGGAACCGCCGCCCTGGG
CTGCCTGGGAAGGACTACTTCCCCGAGCCCGTGACCGTGTCCTGGAACAG
CGGCGCTCTGACCAGCGGAGTGCACACCTTCCCTGCCGTGCTGCAGAGCA
GCGGCCTGTACTCCCTGAGCAGCGTGGTGACCGTGCCAGCAGCAGCCTGG
GCACCCAGACCTACATCTGCAACGTGAACCACAAGCCCTCCAACACCAAG
GTGGACAAGAAGGTGGAGCCTAAGAGCTGCGACAAGACCCACACCTGCCC
TCCCTGCCCCGCCCCGAGCTGCTGGGCGGACCCAGCGTGTTCCTGTTCCC
TCCCAAGCCCAAGGACACCCTGATGATCAGCCGCACCCCCGAGGTGACCT
GCGTGGTGGTGGACGTGAGCCACGAGGACCCCGAGGTGAGTTCAACTGGT
ACGTGGACGGCGTGGAGGTGCACAACGCCAAGACCAAGCCTCGGGAGGAG
CAGTACAACTCCACCTACCGCGTGGTGAGCGTGCTGACCGTGCTGCACCA
GGACTGGCTGAACGGCAGGAGTACAAGTGCAAGGTGAGCAACAAGGCCCT
GCCCGCTCCCATCGAGAAGACCATCAGCAAGGCCAAGGGCCAGCCCCGGG
AGCCTCAGGTGTACACCCTGCCCCCCAGCCGCGACGAGCTGACAAGAACC
AGGTGAGCCTGACCTGCCTGGTGAAGGGCTTCTACCCCTCCGACATCGCC
GTGGAGTGGGAGAGCAACGGCCAGCCTGAGAACAACTACAAGACCACCCC
TCCCGTGCTGGACAGCGACGCAGCTTCTTCCTGTACAGCAAGCTGACCGT
GGACAAGTCCCGGTGGCAGCAGGGCAACGTGTTCAGCTGCAGCGTGATGC
ACGAGGCCCTGCACAACCACTACACCCAGAAGAGCCTGAGCCTGAGCCCG
GATAGTAA.

The one-letter amino acid sequence that corresponds to SEQ ID NO: 35 is

METDTLLLWVLLLWVPGSTGDVVMTQSPLSLPVTLGQPASISCRSSQSLV
NSNGNTFLQWYQQRPGQSPRLLIYKVSLRFSGVPDRFSGSGSGTDFTLKI
SRVEAEDVGVYYCSQSTHVPPTFGGGTVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEQ ID NO: 36 is

ATGGAGACCGACACCCTGCTGCTCTGGGTGCTGCTGCTCTGGGTGCCCGG
CTCCACCGGAGACGTCGTGATGACCCAGTCCCCTCTGTCCCTGCCTGTGA
CCCTGGGACAGCCTGCCTCCATCTCCTCAGATCCTCCCAGTCCCTCGTGA
ACTCCAACGGCAACACCTTCCTGCAGTGGTATCAGCAGCGGCCTGGCCAG
AGCCCCAGACTGCTGATCTACAAGGTGTCCCTGCGGTTCTCCGGCGTGCC
CGACGATTTTCCGGCTCTGGCTCTGGCACCGACTTCACCCTGAAGATCTC
CCGGGTGGAAGCCGAGGACGTGGGCGTGTACTACTGCTCCCAGAGCACCC
ACGTGCCCCCTACATTTGGCGGAGGCACCAAGTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

The one-letter amino acid sequence that corresponds to SEQ ID NO: 37 is

METDTLLLWVLLLWVPGSTGDVVMTQSPLSLPVTLGQPASISCRSRQSLV
NSNGNTFLQWYQQRPGQSPRLLIYKVSLRFSGVPDRFSGSGSGTDFTLKI
SRVEAEDVGVYYCSQSTHVPPTFGGGTVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEQ ID NO: 38 is

ATGGAGACCGACACCCTGCTGCTCTGGGTGCTGCTGCTCTGGGTGCCCGG
CTCCACCGGAGACGTCGTGATGACCCAGTCCCCTCTGTCCCTGCCTGTGA
CCCTGGGACAGCCTGCCTCCATCTCCTCAGATCCAGGCAGTCCCTCGTGA
ACTCCAACGGCAACACCTTCCTGCAGTGGTATCAGCAGCGGCCTGGCCAG
AGCCCCAGACTGCTGATCTACAAGGTGTCCCTGCGGTTCTCCGGCGTGCC
CGACGATTTTCCGGCTCTGGCTCTGGCACCGACTTCACCCTGAAGATCTC
CCGGGTGGAAGCCGAGGACGTGGGCGTGTACTACTGCTCCCAGAGCACCC
ACGTGCCCCCTACATTTGGCGGAGGCACCAAGTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

The one-letter amino acid sequence that corresponds to SEQ ID NO: 39 is

METDTLLLWVLLLWVPGSTGDVVMTQSPLSSPVTLGQPASISCRSSQSLV
NSNGNTFLQWYHQRPGQPPRLLIYKVSLRFSGVPDRFSGSGAGKDFTLKI
SRVEAEDVGVYYCSQSTHVPPTFGQGTLEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC.

The DNA sequence that corresponds to SEQ ID NO: 40 is

ATGGAGACCGACACCCTGCTGCTCTGGGTGCTGCTGCTCTGGGTGCCCGG
CTCCACCGGAGACGTCGTGATGACCCAGTCCCCTCTGTCCAGTCCTGTGA
CCCTGGGACAGCCTGCCTCCATCTCCTCAGATCCTCCCAGTCCCTCGTGA
ACTCCAACGGCAACACCTTCCTGCAGTGGTATCACCAGCGGCCTGGCCAG
CCTCCCAGACTGCTGATCTACAAGGTGTCCCTGCGGTTCTCCGGCGTGCC
CGACGATTTTCCGGCTCTGGCGCTGGCAAGGACTTCACCCTGAAGATCTC
CCGGGTGGAAGCCGAGGACGTGGGCGTGTACTACTGCTCCCAGAGCACCC
ACGTGCCCCCTACATTTGGCCAGGGCACCAACTGGAAATCAAGCGGACCG
TGGCCGCCCCCAGCGTGTTCATCTTCCCTCCCAGCGACGAGCAGCTGAAG
TCTGGCACCGCCAGCGTGGTGTGCCTGCTGAACAACTTCTACCCCCGCGA
GGCCAAGGGCAGTGGAAGGTGGACAACGCCCTGCAGAGCGGCAACAGCCA
GGAGAGCGTGACCGAGCAGGACTCCAAGGACAGCACCTACAGCCTGAGCA
GCACCCTGACCCTGAGCAAGGCCGACTACGAGAAGACAAGGTGTACGCCT
GCGAGGTGACCCACCAGGGACTGTCTAGCCCCGTGACCAAGAGCTTCAAC
CGGGGCGAGTGCTAA.

The one-letter amino acid sequence that corresponds to SEQ ID NO: 47 is

MGWTLVFLFLLSVTAGVHSQVQLLQPGAELVKPGASVKLACKASGYLFTT
YWMHWLKQRPGQGLEWIGEISPTNGRAYYNARFKSEATLTVDKSSNTAYM
QLSSLTSEASAVYYCARSFGNYEFAYWGQGTLVTVSVASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

The one-letter amino acid sequence that corresponds to SEQ ID NO: 48 is

MGWTLVFLFLLSVTAGVHSEVQLLESGAEAKKPGASVKLSCKASGYLFTT
YWMHWVHQAPGQRLEWMGEISPTNGRAYYNARFKSRVTITVDKSASTAYM
ELSSLRSEDTAVYYCARSFGNYEFAYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

The one-letter amino acid sequence that corresponds to SEQ ID NO: 49 is

MGWTLVFLFLLSVTAGVHSQVQLVQSGAEVKKPGASVKVSCKASGYLFTT
YWMHWVRQAPGQRLEWIGEISPTNGRAYYNARFKSRVTITRDTSASTAYM
ELSSLRSEDTAVYYCARSFGNYEFAYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

The one-letter amino acid sequence that corresponds to SEQ ID NO: 50 is

MGWTLVFLFLLSVTAGVHSQVQLVQSGAEVKKPGSSVKVSCKASGYLFTT
YWMHWVRQAPGQGLEWMGEISPTNGRAYYNARFKSRVTITADKSTSTAYM
ELSSLRSEDTAVYYCARSFGNYEFAYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

The one-letter amino acid sequence that corresponds to SEQ ID NO: 51 is

MGWTLVFLFLLSVTAGVHSQVQLVQSGAEVKKPGASVKVSCEASGYLFTT
YWMHWVRQAPGQGLEWMGEISPTNGRAYYNARFKSRVTITRDTSINTAYM
ELSRLRSDDTAVYYCARSFGNYEFAYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK.

The one-letter amino acid sequence that corresponds to SEQ ID NO: 52 is

QVQLLQPGAELVKPGASVKLACKASGYLFTTYWMHWLKQRPGQGLEWIG
EISPTNGRAYYNARFKSEATLTVDKSSNTAYMQLSSLTSEASAVYYCAR
SFGNYEFAYWGQGTLVTVSV.

The one-letter amino acid sequence that corresponds to SEQ ID NO: 53 is

EVQLLESGAEAKKPGASVKLSCKASGYLFTTYWMHWVHQAPGQRLEWMG
EISPTNGRAYYNARFKSRVTITVDKSASTAYMELSSLRSEDTAVYYCAR
SFGNYEFAYWGQGTLVTVSS.

The one-letter amino acid sequence that corresponds to SEQ ID NO: 54 is

QVQLVQSGAEVKKPGASVKVSCKASGYLFTTYWMHWVRQAPGQRLEWIG
EISPTNGRAYYNARFKSRVTITRDTSASTAYMELSSLRSEDTAVYYCAR
SFGNYEFAYWGQGTLVTVSS.

The one-letter amino acid sequence that corresponds to SEQ ID NO: 55 is

QVQLVQSGAEVKKPGSSVKVSCKASGYLFTTYWMHWVRQAPGQGLEWMG
EISPTNGRAYYNARFKSRVTITADKSTSTAYMELSSLRSEDTAVYYCAR
SFGNYEFAYWGQGTLVTVSS.

The one-letter amino acid sequence that corresponds to SEQ ID NO: 56 is

QVQLVQSGAEVKKPGASVKVSCEASGYLFTTYWMHWVRQAPGQGLEWMG
EISPTNGRAYYNARFKSRVTITRDTSINTAYMELSRLRSDDTAVYYCAR
SFGNYEFAYWGQGTLVTVSS.

The one-letter amino acid sequence that corresponds to SEQ ID NO: 57 is

MVSSAQFLGLLLLCFQGTRCDVVMTQTPLSLPVSLGDQASISCRSRQSL
VNSNGNTFLQWYLQKPGQSPKLLIYKVSLRFSGVPDRFSGSGSGTDFTL
KISRVEAEDLGLYFCSQSTHVPPTFGGGTKLEIKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC.

The one-letter amino acid sequence that corresponds to SEQ ID NO: 58 is

MVSSAQFLGLLLLCFQGTRCDIVMTQTPLSLPVTLGQPASISCRSRQSL
VNSNGNTFLQWLQQRPGQPPRLLIYKVSLRFSGVPDRFSGSGAGTDFTL
TISRVEAEDVGIYFCSQSTHVPPTFGQGTKVEIKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC.

The one-letter amino acid sequence that corresponds to SEQ ID NO: 59 is

MVSSAQFLGLLLLCFQGTRCDIVMTQTPLSLSVTPGQPASISCRSRQSL
VNSNGNTFLQWYLQKPGQSPQLLIYKVSLRFSGVPDRFSGSGSGTDFTL
KISRVEPEDVGVYYCSQSTHVPPTFGGGTKVEVKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC.

The one-letter amino acid sequence that corresponds to SEQ ID NO: 60 is

MVSSAQFLGLLLLCFQGTRCDVVMTQSPLSLPVTLGQPASISCRSRQSL
VNSNGNTFLQWFQQRPGQSPRRLIYKVSLRFSGVPDRFSGSGSDTDFTL
RISRVEAEDVGLYYCSQSTHVPPTFGQGTKLEIKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC.

The one-letter amino acid sequence that corresponds to SEQ ID NO: 61 is

MVSSAQFLGLLLLCFQGTRCDIVMTQTPLSLSVTPGQPASISCRSRQSL
VNSNGNTFLQWLLQKPGQPPQLLIYKVSLRFSGVPNRFSGSGSGTDFTL
KISRVEAEDVGLYYCSQSTHVPPTFGGGTKVEIKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC.

The one-letter amino acid sequence that corresponds to SEQ ID NO: 62 is

DVVMTQTPLSLPVSLGDQASISCRSRQSLVNSNGNTFLQWYLQKPGQSP
KLLIYKVSLRFSGVPDRFSGSGSGTDFTLKISRVEAEDLGLYFCSQSTH
VPPTFGGGTKLEIK.

The one-letter amino acid sequence that corresponds to SEQ ID NO: 63 is

DIVMTQTPLSLPVTLGQPASISCRSRQSLVNSNGNTFLQWLQQRPGQPP
RLLIYKVSLRFSGVPDRFSGSGAGTDFTLTISRVEAEDVGIYFCSQSTH
VPPTFGQGTKVEIK.

The one-letter amino acid sequence that corresponds to SEQ ID NO: 64 is

DIVMTQTPLSLSVTPGQPASISCRSRQSLVNSNGNTFLQWYLQKPGQSP
QLLIYKVSLRFSGVPDRFSGSGSGTDFTLKISRVEPEDVGVYYCSQSTH
VPPTFGGGTKVEVK.

The one-letter amino acid sequence that corresponds to SEQ ID NO: 65 is

DVVMTQSPLSLPVTLGQPASISCRSRQSLVNSNGNTFLQWFQQRPGQSP
RRLIYKVSLRFSGVPDRFSGSGSDTDFTLRISRVEAEDVGLYYCSQSTH
VPPTFGQGTKLEIK.

The one-letter amino acid sequence that corresponds to SEQ ID NO: 66 is

DIVMTQTPLSLSVTPGQPASISCRSRQSLVNSNGNTFLQWLLQKPGQPP
QLLIYKVSLRFSGVPNRFSGSGSGTDFTLKISRVEAEDVGLYYCSQSTH
VPPTFGGGTKVEIK.

### Examples

### Example 1: In Vivo Study of the Administration of Anti-Glycation End-Product Antibody

To examine the effects of an anti-glycation end-product antibody, the antibody was administered to the aged CD1(ICR) mouse (Charles River Laboratories), twice daily by intravenous injection, once a week, for three weeks (Days 1, 8 and 15), followed by a 10 week treatment-free period. The test antibody was a commercially available mouse anti-glycation end-product antibody raised against carboxymethyl lysine conjugated with keyhole limpet hemocyanin, the carboxymethyl lysine MAb (Clone 318003) available from R&D Systems, Inc. (Minneapolis, Minn.; catalog no. MAB3247). A control reference of physiological saline was used in the control animals.

Mice referred to as “young” were 8 weeks old, while mice referred to as “old” were 88 weeks (±2 days) old. No adverse events were noted from the administration of the antibody. The different groups of animals used in the study are shown in Table 1.

TABLE 1
The different groups of animals used in the study

| | | | | Number of Animals | |
|---|---|---|---|---|---|
| | | | | Main | Treatment- |
| Group | | | Dose Level | Study | Free |
| No. | Test Material | Mice | (μg/gm/BID/week) | Females | Females |
| 1 | Saline | young | 0 | 20 | — |
| 2 | Saline | old | 0 | 20 | 20 |
| 3 | Antibody | old | 2.5 | 20 | 20 |
| 4 | None | old | 0 | 20 | pre |
| 5 | Antibody | old | 5.0 | 20 | 20 |

— = Not Applicable, Pre = Subset of animals euthanized prior to treatment start for collection of adipose tissue.

p16^{INK4a} mRNA, a marker for senescent cells, was quantified in adipose tissue of the groups by Real Time-qPCR. The results are shown in Table 2. In the table ΔΔCt=ΔCt mean control Group (2) −ΔCt mean experimental Group (1 or 3 or 5); Fold Expression=2^{-ΔΔct}.

TABLE 2
P16 mRNA^{INK4a} quantified in adipose tissue

| Calculation | | | | | | |
|---|---|---|---|---|---|---|
| (unadjusted | Group 2 vs | | Group 2 vs | | Group 2 vs | |
| to Group | Group 1 | | Group 3 | | Group 5 | |
| 4: 5.59) | Group 2 | Group 1 | Group 2 | Group 3 | Group 2 | Group 5 |
| Mean ΔCt | 5.79 | 7.14 | 5.79 | 6.09 | 5.79 | 7.39 |

| | | | |
|---|---|---|---|
| ΔΔCt | −1.35 | −0.30 | −1.60 |
| Fold | 2.55 | 1.23 | 3.03 |
| Expression | | | |

The table above indicates that untreated old mice (Control Group 2) express 2.55-fold more p16^{Ink4a} mRNA than the untreated young mice (Control Group 1), as expected. This was observed when comparing Group 2 untreated old mice euthanized at end of recovery Day 85 to Group 1 untreated young mice euthanized at end of treatment Day 22. When results from Group 2 untreated old mice were compared to results from Group 3 treated old mice euthanized Day 85, it was observed that p16^{Ink4a} mRNA was 1.23-fold higher in Group 2 than in Group 3. Therefore, the level of p16^{Ink4a} mRNA expression was lower when the old mice were treated with 2.5 μg/gram/BID/week of antibody.

When results from Group 2 (Control) untreated old mice were compared to results from Group 5 (5 μg/gram) treated old mice euthanized Day 22, it was observed that p16^{Ink4a} mRNA was 3.03-fold higher in Group 2 (controls) than in Group 5 (5 μg/gram). This comparison indicated that the Group 5 animals had lower levels of p16^{Ink4a} mRNA expression when they were treated with 5.0 μg/gram/BID/week, providing p16^{Ink4a} mRNA expression levels comparable to that of the young untreated mice (i.e. Group 1). Unlike Group 3 (2.5 μg/gram) mice that were euthanized at end of recovery Day 85, Group 5 mice were euthanized at end of treatment Day 22.

These results indicate the antibody administration resulted in the killing of senescent cells.

The mass of the gastrocnemius muscle was also measured, to determine the effect of antibody administration on sarcopenia. The results are provided in Table 3. The results indicate that administration of the antibody increased muscle mass as compared to controls, but only at the higher dosage of 5.0 μg/gm/BID/week.

TABLE 3
Effect of antibody administration on mass of
the gastrocnemius muscle

| | | Absolute weight | Weight relative |
|---|---|---|---|
| | Summary | of Gastrocnemius | to body mass of |
| Group | Information | Muscle | Gastrocnemius Muscle |
| 1 | Mean | 0.3291 | 1.1037 |
| | SD | 0.0412 | 0.1473 |
| | N | 20 | 20 |
| 2 | Mean | 0.3304 | 0.7671 |
| | SD | 0.0371 | 0.1246 |
| | N | 20 | 20 |
| 3 | Mean | 0.3410 | 0.7706 |
| | SD | 0.0439 | 0.0971 |
| | N | 19 | 19 |
| 5 | Mean | 0.4074 | 0.9480 |
| | SD | 0.0508 | 0.2049 |
| | N | 9 | 9 |

These results demonstrate that administration of antibodies that bind to AGEs of a cell resulted in a reduction of cells expressing p16^{Ink4a}, a biomarker of senescence. The data show that reducing senescent cells leads directly to an increase in muscle mass in aged mice. These results indicate that the loss of muscle mass, a classic sign of sarcopenia, can be treated by administration of antibodies that bind to AGEs of a cell. The results suggest that administration of the antibodies would be effective in treating premature aging by removing senescent cells.

### Example 2: Affinity and Kinetics of Test Antibody

The affinity and kinetics of the test antibody used in Example 1 were analyzed using Nα,Nα-bis(carboxymethyl)-L-lysine trifluoroacetate salt (Sigma-Aldrich, St. Louis, Mo.) as a model substrate for an AGE-modified protein of a cell. Label-free interaction analysis was carried out on a BIACORE™ T200 (GE Healthcare, Pittsburgh, Pa.), using a Series S sensor chip CM5 (GE Healthcare, Pittsburgh, Pa.), with Fc1 set as blank, and Fc2 immobilized with the test antibody (molecular weight of 150,000 Da). The running buffer was a HBS-EP buffer (10 mM HEPES, 150 mM NaCl, 3 mM EDTA and 0.05% P-20, pH of 7.4), at a temperature of 25° C. Software was BIACORE™ T200 evaluation software, version 2.0. A double reference (Fc2-1 and only buffer injection), was used in the analysis, and the data was fitted to a Langmuir 1:1 binding model.

TABLE 4
Experimental set-up of affinity and kinetics analysis
Association and dissociation

| Flow path | Fc1 and Fc2 |
|---|---|
| Flow rate (μl/min.) | 30 |
| Association time (s) | 300 |
| Dissociation time (s) | 300 |
| Sample concentration (μM) | 20 − 5 − 1.25 (×2) − 0.3125 − 0.078 − 0 |

A graph of the response versus time is illustrated in FIG. 1. The following values were determined from the analysis: kₐ (1/Ms)=1.857×10³; k_{d} (1/s)=6.781×10⁻³; K_{D} (M)=3.651×10⁻⁶; Rₘₐₓ (RU)=19.52; and Chi²=0.114. Because the Chi² value of the fitting is less than 10% of Rₘₐₓ, the fit is reliable.

### Example 3: Construction and Production of Murine Anti-AGE IgG2b Antibody and Chimeric Anti-AGE IgG1 Antibody

Murine and chimeric human anti-AGE antibodies were prepared. The DNA sequence of murine anti-AGE antibody IgG2b heavy chain is shown in SEQ ID NO: 12. The DNA sequence of chimeric human anti-AGE antibody IgG1 heavy chain is shown in SEQ ID NO: 13. The DNA sequence of murine anti-AGE antibody kappa light chain is shown in SEQ ID NO: 14. The DNA sequence of chimeric human anti-AGE antibody kappa light chain is shown in SEQ ID NO: 15. The gene sequences were synthesized and cloned into high expression mammalian vectors. The sequences were codon optimized. Completed constructs were sequence confirmed before proceeding to transfection.

HEK293 cells were seeded in a shake flask one day before transfection, and were grown using serum-free chemically defined media. The DNA expression constructs were transiently transfected into 0.03 liters of suspension HEK293 cells. After 20 hours, cells were sampled to obtain the viabilities and viable cell counts, and titers were measured (OCTET® QKe, ForteBio). Additional readings were taken throughout the transient transfection production runs. The cultures were harvested on day 5, and an additional sample for each was measured for cell density, viability and titer.

The conditioned media for murine and chimeric anti-AGE antibodies were harvested and clarified from the transient transfection production runs by centrifugation and filtration. The supernatants were run over a Protein A column and eluted with a low pH buffer. Filtration using a 0.2 μm membrane filter was performed before aliquoting. After purification and filtration, the protein concentrations were calculated from the OD280 and the extinction coefficient. A summary of yields and aliquots is shown in Table 5:

TABLE 5
Yields and aliquots

| | Concentration | Volume | No. | Total |
|---|---|---|---|---|
| Protein | (mg/mL) | (mL) | of vials | Yield (mg) |
| Murine anti-AGE | 0.08 | 1.00 | 3 | 0.24 |
| Chimeric anti-AGE | 0.23 | 1.00 | 3 | 0.69 |

Antibody purity was evaluated by capillary electrophoresis sodium-dodecyl sulfate (CE-SDS) analysis using LabChip® GXII, (PerkinElmer).

### Example 4: Binding of Murine (Parental) and Chimeric Anti-AGE Antibodies

The binding of the murine (parental) and chimeric anti-AGE antibodies described in Example 3 was investigated by a direct binding ELISA. An anti-carboxymethyl lysine (CML) antibody (R&D Systems, MAB3247) was used as a control. CML was conjugated to KLH (CML-KLH) and both CML and CML-KLH were coated overnight onto an ELISA plate. HRP-goat anti-mouse Fc was used to detect the control and murine (parental) anti-AGE antibodies. HRP-goat anti-human Fc was used to detect the chimeric anti-AGE antibody.

The antigens were diluted to 1 μg/mL in 1× phosphate buffer at pH 6.5. A 96-well microtiter ELISA plate was coated with 100 μL/well of the diluted antigen and let sit at 4° C. overnight. The plate was blocked with 1×PBS, 2.5% BSA and allowed to sit for 1-2 hours the next morning at room temperature. The antibody samples were prepared in serial dilutions with 1×PBS, 1% BSA with the starting concentration of 50 μg/mL. Secondary antibodies were diluted 1:5,000. 100 μL of the antibody dilutions was applied to each well. The plate was incubated at room temperature for 0.5-1 hour on a microplate shaker. The plate was washed 3 times with 1×PBS. 100 μL/well diluted HRP-conjugated goat anti-human Fc secondary antibody was applied to the wells. The plate was incubated for 1 hour on a microplate shaker. The plate was then washed 3 times with 1×PBS. 100 μL HRP substrate TMB was added to each well to develop the plate. After 3-5 minutes elapsed, the reaction was terminated by adding 100 μL of 1N HCl. A second direct binding ELISA was performed with only CML coating. The absorbance at OD450 was read using a microplate reader.

The OD450 absorbance raw data for the CML and CML-KLH ELISA is shown in the plate map below. 48 of the 96 wells in the well plate were used. Blank wells in the plate map indicate unused wells.

Plate Map of CML and CML-KLH ELISA:

| Conc. | | | | | | | |
|---|---|---|---|---|---|---|---|
| (μg/mL) | 1 | 2 | 3 | 4 | 5 | 6 | 7 |
| 50 | 0.462 | 0.092 | 0.42 | | 1.199 | 0.142 | 1.852 |
| 16.67 | 0.312 | 0.067 | 0.185 | | 0.31 | 0.13 | 0.383 |
| 5.56 | 0.165 | 0.063 | 0.123 | | 0.19 | 0.115 | 0.425 |
| 1.85 | 0.092 | 0.063 | 0.088 | | 0.146 | 0.099 | 0.414 |
| 0.62 | 0.083 | 0.072 | 0.066 | | 0.108 | 0.085 | 0.248 |
| 0.21 | 0.075 | 0.066 | 0.09 | | 0.096 | 0.096 | 0.12 |
| 0.07 | 0.086 | 0.086 | 0.082 | | 0.098 | 0.096 | 0.098 |
| 0 | 0.09 | 0.085 | 0.12 | | 0.111 | 0.083 | 0.582 |
| | R&D | Parental | Chimeric | | R&D | Parental | Chimeric |
| | Positive | Anti-AGE | Anti-AGE | | Positive | Anti-AGE | Anti-AGE |
| | Control | | | | Control | | |

| | |
|---|---|
| CML-KLH Coat | CML Coat |

The OD450 absorbance raw data for the CML-only ELISA is shown in the plate map below. 24 of the 96 wells in the well plate were used. Blank wells in the plate map indicate unused wells.

Plate Map of CML-Only ELISA:

| Conc. | | | | | | | |
|---|---|---|---|---|---|---|---|
| (μg/mL) | 1 | 2 | 3 | 4 | 5 | 6 | 7 |
| 50 | 1.913 | 0.165 | 0.992 | | | | |
| 16.66667 | 1.113 | 0.226 | 0.541 | | | | |
| 5.555556 | 0.549 | 0.166 | 0.356 | | | | |
| 1.851852 | 0.199 | 0.078 | 0.248 | | | | |
| 0.617284 | 0.128 | 0.103 | 0.159 | | | | |
| 0.205761 | 0.116 | 0.056 | 0.097 | | | | |
| 0.068587 | 0.073 | 0.055 | 0.071 | | | | |
| 0 | 0.053 | 0.057 | 0.06 | | | | |
| | R&D | Parental | Chimeric | | | | |
| | Positive | Anti- | Anti- | | | | |
| | Control | AGE | AGE | | | | |

The control and chimeric anti-AGE antibodies showed binding to both CML and CML-KLH. The murine (parental) anti-AGE antibody showed very weak to no binding to either CML or CML-KLH. Data from repeated ELISA confirms binding of the control and chimeric anti-AGE to CML. All buffer control showed negative signal.

### Example 5: Humanized Antibodies

Humanized antibodies were designed by creating multiple hybrid sequences that fuse select parts of the parental (mouse) antibody sequence with the human framework sequences. Acceptor frameworks were identified based on the overall sequence identity across the framework, matching interface position, similarly classed CDR canonical positions, and presence of N-glycosylation sites that would have to be removed. Three humanized light chains and three humanized heavy chains were designed based on two different heavy and light chain human acceptor frameworks. The amino acid sequences of the heavy chains are shown in SEQ ID NO: 29, 31 and 33, which are encoded by the DNA sequences shown in SEQ ID NO: 30, 32 and 34, respectively. The amino acid sequences of the light chains are shown in SEQ ID NO: 35, 37 and 39, which are encoded by the DNA sequences shown in SEQ ID NO: 36, 38 and 40, respectively. The humanized sequences were methodically analyzed by eye and computer modeling to isolate the sequences that would most likely retain antigen binding. The goal was to maximize the amount of human sequence in the final humanized antibodies while retaining the original antibody specificity. The light and heavy humanized chains could be combined to create nine variant fully humanized antibodies.

The three heavy chains and three light chains were analyzed to determine their humanness. Antibody humanness scores were calculated according to the method described in Gao, S. H., et al., “Monoclonal antibody humanness score and its applications”, BMC Biotechnology, 13:55 (Jul. 5, 2013). The humanness score represents how human-like an antibody variable region sequence looks. For heavy chains a score of 79 or above is indicative of looking human-like; for light chains a score of 86 or above is indicative of looking human-like. The humanness of the three heavy chains, three light chains, a parental (mouse) heavy chain and a parental (mouse) light chain are shown below in Table 6:

TABLE 6
Antibody humanness

| Antibody | Humanness (Framework + CDR) |
|---|---|
| Parental (mouse) heavy chain | 63.60 |
| Heavy chain 1 (SEQ ID NO: 29) | 82.20 |
| Heavy chain 2 (SEQ ID NO: 31) | 80.76 |
| Heavy chain 3 (SEQ ID NO: 33) | 81.10 |
| Parental (mouse) light chain | 77.87 |
| Light chain 1 (SEQ ID NO: 35) | 86.74 |
| Light chain 2 (SEQ ID NO: 37) | 86.04 |
| Light chain 3 (SEQ IN NO: 39) | 83.57 |

Full-length antibody genes were constructed by first synthesizing the variable region sequences. The sequences were optimized for expression in mammalian cells. These variable region sequences were then cloned into expression vectors that already contain human Fc domains; for the heavy chain, the IgG1 was used.

Small scale production of humanized antibodies was carried out by transfecting plasmids for the heavy and light chains into suspension HEK293 cells using chemically defined media in the absence of serum. Whole antibodies in the conditioned media were purified using MabSelect SuRe Protein A medium (GE Healthcare).

Nine humanized antibodies were produced from each combination of the three heavy chains having the amino acid sequences shown in SEQ ID NO: 29, 31 and 33 and three light chains having the amino acid sequences shown in SEQ ID NO: 35, 37 and 39. A comparative chimeric parental antibody was also prepared. The antibodies and their respective titers are shown below in Table 7:

TABLE 7
Antibody titers

| Antibody | Titer (mg/L) |
|---|---|
| Chimeric parental | 23.00 |
| SEQ ID NO: 29 + SEQ ID NO: 35 | 24.67 |
| SEQ ID NO: 29 + SEQ ID NO: 37 | 41.67 |
| SEQ ID NO: 29 + SEQ ID NO: 39 | 29.67 |
| SEQ ID NO: 31 + SEQ ID NO: 35 | 26.00 |
| SEQ ID NO: 31 + SEQ ID NO: 37 | 27.33 |
| SEQ ID NO: 31 + SEQ ID NO: 39 | 35.33 |
| SEQ ID NO: 33 + SEQ ID NO: 35 | 44.00 |
| SEQ ID NO: 33 + SEQ ID NO: 37 | 30.33 |
| SEQ ID NO: 33 + SEQ ID NO: 39 | 37.33 |

The binding of the humanized antibodies may be evaluated, for example, by dose-dependent binding ELISA or cell-based binding assay.

### Example 6: An AGE-RNAse Containing Vaccine in a Human Subject

AGE-RNAse is prepared by incubating RNAse in a phosphate buffer solution containing 0.1-3 M glucose, glucose-6-phosphate, fructose or ribose for 10-100 days. The AGE-RNAse solution is dialyzed and the protein content is measured. Aluminum hydroxide or aluminum phosphate, as an adjuvant, is added to 100 μg of the AGE-RNAse. Formaldehyde or formalin is added as a preservative to the preparation. Ascorbic acid is added as an antioxidant. The vaccine also includes phosphate buffer to adjust the pH and glycine as a protein stabilizer. The composition is injected intravenously into a subject with progeria.

### Example 7: Injection Regimen for an AGE-RNAse Containing Vaccine in a Human Subject

The same vaccine as described in Example 6 is injected intravenously into a subject who has been identified as experiencing premature aging based on a diagnosis of early onset Alzheimer's disease. The titer of antibodies to AGE-RNAse is determined by ELISA after two weeks. Additional injections are performed after three weeks and six weeks, respectively. Further titer determination is performed two weeks after each injection.

### Example 8: An AGE-Hemoglobin Containing Vaccine in a Human Subject

AGE-hemoglobin is prepared by incubating human hemoglobin in a phosphate buffer solution containing 0.1-3 M glucose, glucose-6-phosphate, fructose or ribose for 10-100 days. The AGE-hemoglobin solution is dialyzed and the protein content is measured. All vaccine components are the same as in Example 6, except AGE-hemoglobin is substituted for AGE-RNAse. Administration is carried out as in Example 6, or as in Example 7.

### Example 9: An AGE-Human Serum Albumin Containing Vaccine in a Human subject

AGE-human serum albumin is prepared by incubating human serum albumin in a phosphate buffer solution containing 0.1-3 M glucose, glucose-6-phosphate, fructose or ribose for 10-100 days. The AGE-human serum albumin solution is dialyzed and the protein content is measured. All vaccine components are the same as in Example 6, except AGE-human serum albumin is substituted for AGE-RNAse. Administration is carried out as in Example 6, or as in Example 7.

### Example 10: Carboxymethyllysine-Modified Protein Vaccine for a Human Subject

A vaccine is prepared by combining a carboxymethyllysine-modified protein as an AGE antigen, aluminum hydroxide as an adjuvant, formaldehyde as a preservative, ascorbic acid as an antioxidant, a phosphate buffer to adjust the pH of the vaccine and glycine as a protein stabilizer. The vaccine is injected subcutaneously into a subject who developed cardiovascular disease after receiving ionizing radiation therapy.

### Example 11: Carboxyethyllysine-Modified Peptide Vaccine for a Human Subject

A vaccine is prepared by combining a carboxyethyllysine-modified peptide conjugated to KLH as an AGE antigen, aluminum hydroxide as an adjuvant, formaldehyde as a preservative, ascorbic acid as an antioxidant, a phosphate buffer to adjust the pH of the vaccine and glycine as a protein stabilizer. The vaccine is injected subcutaneously into a subject with chronic kidney disease who had been exposed to dioxin.

### Example 12: In Vivo Study of the Administration of a Carboxymethyl Lysine Monoclonal Antibody

The effect of a carboxymethyl lysine antibody on tumor growth, metastatic potential and cachexia was investigated. In vivo studies were carried out in mice using a murine breast cancer tumor model. Female BALB/c mice (BALB/cAnNCrl, Charles River) were eleven weeks old on Day 1 of the study.

4T1 murine breast tumor cells (ATCC CRL-2539) were cultured in RPMI 1640 medium containing 10% fetal bovine serum, 2 mM glutamine, 25 μg/mL gentamicin, 100 units/mL penicillin G Na and 100 μg/mL streptomycin sulfate. Tumor cells were maintained in tissue culture flasks in a humidified incubator at 37° C. in an atmosphere of 5% CO₂ and 95% air.

The cultured breast cancer cells were then implanted in the mice. 4T1 cells were harvested during log phase growth and re-suspended in phosphate buffered saline (PBS) at a concentration of 1×10⁶ cells/mL on the day of implant. Tumors were initiated by subcutaneously implanting 1×10⁵ 4 T1 cells (0.1 mL suspension) into the right flank of each test animal. Tumors were monitored as their volumes approached a target range of 80-120 mm³. Tumor volume was determined using the formula: tumor volume=(tumor width)²(tumor length)/2. Tumor weight was approximated using the assumption that 1 mm³ of tumor volume has a weight of 1 mg. Thirteen days after implantation, designated as Day 1 of the study, mice were sorted into four groups (n=15/group) with individual tumor volumes ranging from 108 to 126 mm³ and a group mean tumor volume of 112 mm³. The four treatment groups are shown in Table 8 below:

TABLE 8
Treatment groups

| Group | Description | Agent | Dosing (μg/g) |
|---|---|---|---|
| 1 | Control | phosphate buffered | N/A |
| | | saline (PBS) | |
| 2 | Low-dose | carboxymethyl lysine | 5 |
| | | monoclonal antibody | |
| 3 | High-dose | carboxymethyl lysine | 10 |
| | | monoclonal antibody | |
| 4 | Observation only | None | N/A |

An anti-carboxymethyl lysine monoclonal antibody was used as a therapeutic agent. 250 mg of carboxymethyl lysine monoclonal antibody was obtained from R&D Systems (Minneapolis, Minn.). Dosing solutions of the carboxymethyl lysine monoclonal antibody were prepared at 1 and 0.5 mg/mL in a vehicle (PBS) to provide the active dosages of 10 and 5 μg/g, respectively, in a dosing volume of 10 mL/kg. Dosing solutions were stored at 4° C. protected from light.

All treatments were administered intravenously (i.v.) twice daily for 21 days, except on Day 1 of the study where the mice were administered one dose. On Day 19 of the study, i.v. dosing was changed to intraperitoneal (i.p.) dosing for those animals that could not be dosed i.v. due to tail vein degradation. The dosing volume was 0.200 mL per 20 grams of body weight (10 mL/kg), and was scaled to the body weight of each individual animal.

The study continued for 23 days. Tumors were measured using calipers twice per week. Animals were weighed daily on Days 1-5, then twice per week until the completion of the study. Mice were also observed for any side effects. Acceptable toxicity was defined as a group mean body weight loss of less than 20% during the study and not more than 10% treatment-related deaths. Treatment efficacy was determined using data from the final day of the study (Day 23).

The ability of the anti-carboxymethyl lysine antibody to inhibit tumor growth was determined by comparing the median tumor volume (MTV) for Groups 1-3. Tumor volume was measured as described above. Percent tumor growth inhibition (% TGI) was defined as the difference between the MTV of the control group (Group 1) and the MTV of the drug-treated group, expressed as a percentage of the MTV of the control group. % TGI may be calculated according to the formula: % TGI=(1−MTV_{treated}/MTV_{control})×100.

The ability of the anti-carboxymethyl lysine antibody to inhibit cancer metastasis was determined by comparing lung cancer foci for Groups 1-3. Percent inhibition (% Inhibition) was defined as the difference between the mean count of metastatic foci of the control group and the mean count of metastatic foci of a drug-treated group, expressed as a percentage of the mean count of metastatic foci of the control group. % Inhibition may be calculated according to the following formula: % Inhibition=(1−Mean Count of Foci_{treated}/Mean Count of Foci_{control})×100.

The ability of the anti-carboxymethyl lysine antibody to inhibit cachexia was determined by comparing the weights of the lungs and gastrocnemius muscles for Groups 1-3. Tissue weights were also normalized to 100 g body weight.

Treatment efficacy was also evaluated by the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study.

Statistical analysis was carried out using Prism (GraphPad) for Windows 6.07. Statistical analyses of the differences between Day 23 mean tumor volumes (MTVs) of two groups were accomplished using the Mann-Whitney U test. Comparisons of metastatic foci were assessed by ANOVA-Dunnett. Normalized tissue weights were compared by ANOVA. Two-tailed statistical analyses were conducted at significance level P=0.05. Results were classified as statistically significant or not statistically significant.

The results of the study are shown below in Table 9:

TABLE 9
Results

| | | | | % | | | Gastroc. | |
|---|---|---|---|---|---|---|---|---|
| | | | | In- | | | weight/ | Lung weight/ |
| | MTV | % | Lung | hi- | | | normalized | normalized |
| Group | (mm³) | TGI | foci | bition | PR | CR | (mg) | (mg) |
| 1 | 1800 | N/A | 70.4 | N/A | 0 | 0 | 353.4/19.68 | 2799.4/292.98 |
| 2 | 1568 | 13% | 60.3 | 14% | 0 | 0 | 330.4/21.62 | 2388.9/179.75 |
| 3 | 1688 | 6% | 49.0 | 30% | 0 | 0 | 398.6/24.91 | 2191.6/214.90 |

All treatment regimens were acceptably tolerated with no treatment-related deaths. The only animal deaths were non-treatment-related deaths due to metastasis. The % TGI trended towards significance (P>0.05, Mann-Whitney) for the 5 μg/g (Group 2) and 10 μg/g treatment group (Group 3). The % Inhibition trended towards significance (P>0.05, ANOVA-Dunnett) for the 5 μg/g treatment group. The % Inhibition was statistically significant (P≤0.01, ANOVA-Dunnett) for the 10 μg/g treatment group. The ability of the carboxymethyl lysine antibody to treat cachexia trended towards significance (P>0.05, ANOVA) based on a comparison of the organ weights of the lung and gastrocnemius between treatment groups and the control group. The results indicate that administration of an anti-carboxymethyl lysine monoclonal antibody is able to reduce cancer metastases. This data provides additional evidence that in vivo administration of anti-AGE antibodies can provide therapeutic benefits safely and effectively.

### Example 13: Development of Symptoms which Mimic Premature Aging Due to Ionizing Radiation Study

In vivo studies are carried out in mice to study the effect of treatment with anti-AGE antibodies and vaccination with AGE-KLH on symptoms which mimic premature aging induced by ionizing radiation exposure. Localized development of osteoarthritis will be monitored. Male C57/BL6 mice are 8-10 weeks old on Day 1 of the study. The mice are separated into five treatment groups: (1) control; (2) vehicle only administered intravenously; (3) anti-AGE antibody at 10 μg/g dose administered intravenously; (4) anti-AGE antibody at 10 μg/g dose administered intra-articularly; and (5) 10 μg AGE-KLH administered as a vaccine intraperitoneally.

Osteoarthritis is induced in Groups 2-5 by medial exposing the right hind leg to ionizing radiation. Group 1 is a control where the right hind leg is not irradiated.

Dosing begins one week after the surgery. For Groups 2-5, the dosing volume is 0.200 mL per 20 grams of body weight (10 mL/kg), and is scaled to the body weight of each individual animal. Group 2 receives phosphate-buffered saline (PBS) delivered intravenously. Group 3 receives 10 μg/g of an anti-AGE antibody twice daily for 21 days delivered intravenously. Group 4 receives 10 μg/g of an anti-AGE antibody twice daily for 21 days delivered intra-articularly into the right hind knee. Group 5 receives 10 μg of AGE-KLH in Freunds complete adjuvant intraperitoneally one week prior to exposure to ionizing radiation, followed by a 10 μg/g booster injection of the vaccine four weeks after irradiation.

All Groups are monitored daily for morbidity/mortality and are evaluated daily with attention to effects on locomotion and altered gait. Osteoarthritis is measured in all groups by dynamic weight bearing (DMB) testing.

The animals in Groups 1 and 5 are sacrificed at week 16. For Group 5, the blood is collected for an antibody titer assay, such as the THERMOFISHER® EASY-TITER® Mouse IgG Assay, to determine the titer of antibody in the mice specific for anti-AGE antibodies. An equal number of animals in Groups 2-4 are sacrificed at weeks 4, 8 and 16. Half of the mice in each sacrificed group are analyzed for histology and half are analyzed for p16INK4a qRT PCR. p16INK4a is measured in articular cartilage (chondrocytes) of the animals sacrificed. The p16INK4a qRT PCR is preserved for qRT PCR analysis.

Osteoarthritis is also measured by evaluating samples of the knee joints. Sample of the right and left whole knee joints from all mice are collected and fixed in 10% NBF, then decalcified and embedded in paraffin wax. Three non-consecutive coronal sections are taken for the right knee joint and another three non-consecutive coronal sections are taken for the left knee joint for each staining, providing 6 slides per animal for each stain for a total of 12 slides per animal. The sections are scored for disease severity (cartilage/bone with osteophytes and synovial membrane) by a board certified veterinary pathologist using a semi-quantitative grading system. Scores are reported with statistical analysis.

The anti-AGE antibody will specifically bind to senescent cells and allow the immune system to destroy those cells. Similarly, vaccination with an AGE-KLH antigen will allow the murine immune system to target and remove senescent cells. Killing and removing senescent cells will prevent the development of osteoarthritis and other symptoms which mimic premature aging that would result from exposure to ionizing radiation.

### Example 14: Development of Symptoms which Mimic Premature Aging Due to Ionizing Radiation and Burn Injury Study

In vivo studies are carried out in mice to study the effect of administration of an anti-AGE antibody and vaccination against AGE antigens on symptoms which mimic premature aging induced by ionizing radiation exposure and burn injury. Localized development of pulmonary inflammation will be monitored.

40 mice are organized into four groups, A, B, C and D, of 10 mice per group. Each mouse in Group A is immunized subcutaneously immediately prior to injury with 200 μL of a 1:1 emulsion of Freunds complete adjuvant (Sigma Aldrich) and a 600 μL aliquot of CML adducted keyhole limpet hemocyanin (Biosynthesis) diluted to 400 μg per milliliter in a sterile endotoxin-free PBS. The 10 mice of Group B receive a subcutaneous injection of 800 μL of endotoxin-free PBS solution post wound closure. Group C receives an injection of 10 μg per gram anti-CML antibody. Mice in Group D receive an intradermal injection of endotoxin-free PBS.

All mice are exposed to 5 Gy of total body ionizing radiation by exposure to a ¹³⁷C source at an emission rate of 74.3 cGy (see Palmer, J. L. et al. for additional details). One hour after radiation injury all mice are anesthetized intraperitoneally with a mixture of ketamine (100 mg per kg) and xylazine (10 mg/kg). The dorsal surfaces of the mice are shaved with animal clippers. Each is then placed into a plastic template with an opening allowing 15% total body surface area on their dorsum to be exposed. A scald injury is achieved by immersing the animals in a 95 degrees centigrade water bath for 7 seconds. The mice are dried immediately after exposure to the water to prevent further scalding. All mice receive 1.0 ml of warmed 0.9% saline interperitoneally immediately after exposure the burn injury to compensate for fluid loss and body temperature is maintained by placing their cages on heating pads while the mice recover from the anesthesia. At 48 hours post injury, all mice are sacrificed. Samples of skin from the site of burn injury and unwounded skin are harvested and fixed in 10% buffered formalin, processed and embedded in paraffin. Paraffin sections are subjected to masons trichrome staining parentheses (see Wilgus, T. A. et al. for additional details) and the width of each scar is measured using a stage micrometer. Sections from the lungs of each mouse are stained with hematoxylin and eosin and scored for the presence and count of neutrophil amounts per alveolus.

Mice in Groups A and C will exhibit 50% less scarring and 50% less neutrophils than mice in Groups B and D. Immunization with an AGE antigen (Group A) will allow the murine immune system to target and remove senescent cells. Similarly, administration of an anti-AGE antibody (Group C) will specifically bind to senescent cells and allow the immune system to destroy those cells. Killing and removing senescent cells will prevent the development of pulmonary inflammation and other symptoms which mimic premature aging that would result from exposure to ionizing radiation and burn injury.

### Example 15: Radiation-Induced Senescence and Treatment with Dasatinib and Quercetin

A research group investigated radiation-induced senescence and the removal of senescent cells using the senolytic agents dasatinib and quercetin (Zhu, Y. et al., “The Achilles' heel of senescent cells: from transcriptome to senolytic drugs”, Aging Cell, Vol. 14, pp. 644-658 (2015)). The results are summarized below.

An in vitro study demonstrated that exposure to ionizing radiation alters gene expression in senescent cells. Preadipocytes (fat cell progenitors) were isolated from human subjects and exposed to 10 Gy of ionizing radiation or were sham-irradiated. Gene expression was measured 25 days after radiation exposure. Senescent cells exhibited substantially different gene expression as compared to non-senescent cells, including up-regulation of negative regulators of apoptosis and anti-apoptotic gene sets.

An in vivo study in a mouse model demonstrated that senolytic agents can eliminate senescent cells and provide long-term benefits. Mice had one leg exposed to 10 Gy of radiation with the rest of the body shielded while control mice were sham-irradiated. 12 weeks after radiation exposure the hair on the irradiated limb turned gray and the animals exhibited reduced treadmill exercise capacity, which are signs of premature aging induced by radiation exposure. The mice were then administered a single dose of dasatinib and quercetin (D+Q) or a vehicle-only control. Mice that received a single dose of D+Q exhibited increased exercise time, distance and total work performed to exhaustion on the treadmill 5 days after administration. The treated mice also had reduced senescent markers in muscle and inguinal fat. 7 months following D+Q administration, mice that had been irradiated and treated with a single dose of D+Q exhibited significantly better treadmill exercise capacity as compared to vehicle-treated controls, and had endurance that was essentially identical to the sham-irradiated controls. A single administration of D+Q to sham-irradiated controls had no effect on endurance as compared to vehicle-treated controls 7 months following administration.

These results confirm that radiation exposure leads to senescence both in vitro and in vivo. The in vivo study demonstrates that symptoms of premature aging resulting from radiation exposure can be ameliorated by removal of senescent cells using D+Q.

### Example 16: Chemical Exposure-Induced Senescence and Treatment with Elimination of Senescent Cells Using Genetically-Engineered Mechanisms or ABT-263

A research group investigated therapy-induced senescence (TIS) resulting from chemotherapeutic agents and the removal of senescent cells in a transgenic mouse model (Demaria, M., et al., “Cellular senescence promotes adverse effects of chemotherapy and cancer relapse”, Cancer Discovery, Vol. 7, No. 2, pp. 165-176 (2017)). All in vivo experiments involved the transgenic mouse model p16-3MR, which was specifically engineered to facilitate detection of senescent cells by bioluminescence and elimination of senescent cells by administration of the otherwise-benign antiviral medication ganciclovir (GCV). The results are summarized below.

In vitro and in vivo studies demonstrated that exposure to chemotherapeutic agents induces cellular senescence. In the in vitro study, murine embryonic fibroblasts, murine dermal fibroblasts and human dermal fibroblasts were treated with doxorubicin or paclitaxel. The cells exhibited symptoms of cellular senescence including increased senescence-associated β-galactosidase (SA-β-gal) activity, reduced DNA synthesis, elevated levels of mRNAs encoding p^{16INK4a}, elevated levels of senescence-associated secretory phenotype (SASP) components, elevated levels of p21 and reduced expression of LaminB1. In the in vivo study, mice were administered doxorubicin, paclitaxel, temozolomide or cisplatin. Administration of the chemotherapeutic agents induced senescence in various cell types including keratinocytes, endothelial cells, fibroblasts and smooth muscle cells. Paclitaxel, temozolomide and cisplatin were found to result in elevated p^{16INK4a} expression in the skin.

Multiple in vivo studies demonstrated that elimination of senescent cells by induction of senescent cell elimination in a genetically modified mouse model or by administration of the senolytic agent ABT-263 can effectively treat symptoms resulting from exposure to chemotherapeutic agents. One in vivo study examined inflammation, bone marrow recovery and heart function. Genetically engineered mice were treated with doxorubicin to induce senescence followed by treatment with GCV (to induce elimination of senescent cells). The administration of doxorubicin resulted in increased expression of SASP factor genes associated with inflammation, reduction of hematopoietic progenitor cell (HPC) function and reduction in cardiac function. Elimination of senescent cells reduced circulating inflammatory factors, promoted the functional recovery of HPCs and prevented cardiac dysfunction.

A second in vivo study examined cancer spread and relapse. Genetically engineered mice were treated with doxorubicin after an injection of breast cancer cells (MMTV-PyMT). Mice that received doxorubicin followed by GCV (to induce elimination of senescent cells) exhibited increased survival and reduced cancer metastases as compared to mice that received doxorubicin followed by vehicle-only administration. Other test subjects received surgical removal of palpable tumors prior to treatment. Surgically-treated mice that received GCV (to induce elimination of senescent cells) had smaller tumor growth, reduced cancer metastases and fewer metastatic foci as compared to surgically-treated mice that received doxorubicin followed by vehicle-only administration. Similar results were obtained when senescent cells were eliminated by administration of ABT-263.

A third in vivo study examined chemotherapy-induced fatigue (asthenia). Genetically engineered mice were treated with doxorubicin or paclitaxel to induce senescence followed by the administration of GCV (to induce elimination of senescent cells) or ABT-263. The administration of doxorubicin or paclitaxel resulted in chemotherapy-induced fatigue, as measured by running activity, and decline in strength. Elimination of senescent cells nearly reversed the decline in running activity and improved the loss of strength.

These results confirm that exposure to chemotherapeutic agents leads to senescence both in vitro and in vivo. The in vivo studies demonstrate that symptoms of premature aging resulting from chemical exposure can be ameliorated by elimination of senescent cells. The in vivo studies also establish that the beneficial results may be achieved by inducing the elimination of senescent cells through genetically-engineered mechanisms or by administration of the senolytic agent ABT-263.

### Example 17: Fluorescence Microscopy Study of Chemical Exposure-Induced Senescence

Cells from the pancreatic cancer PANG-1 cell line were treated with 12.5 μM etoposide, a chemotherapeutic agent, for 24 hours to induce senescence. Control cells were treated with dimethyl sulfoxide (DMSO) vehicle for 24 hours. The cells were then stained with a senescence β-galactosidase staining kit, an anti-AGE antibody conjugated to green fluorescent protein (GFP) or an anti-AGE antibody conjugated to GFP and 4′,6-diamidino-2-phenylindole (DAPI). GFP-stained cells appear green and DAPI-stained cells appear blue under fluorescence microscopy.

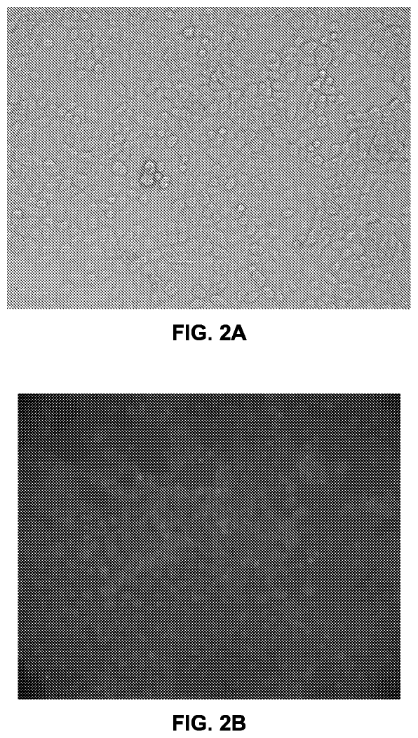

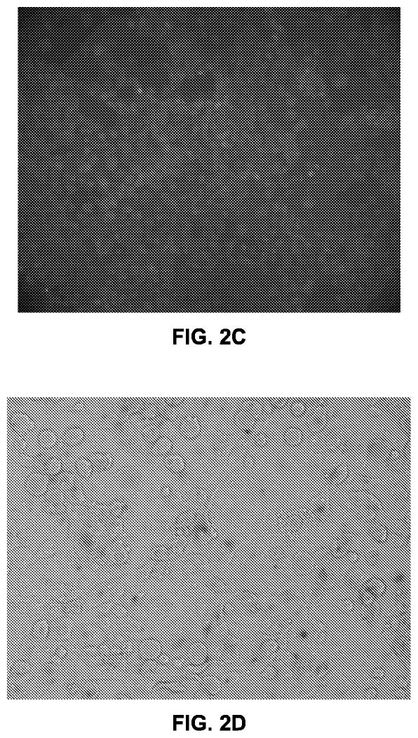

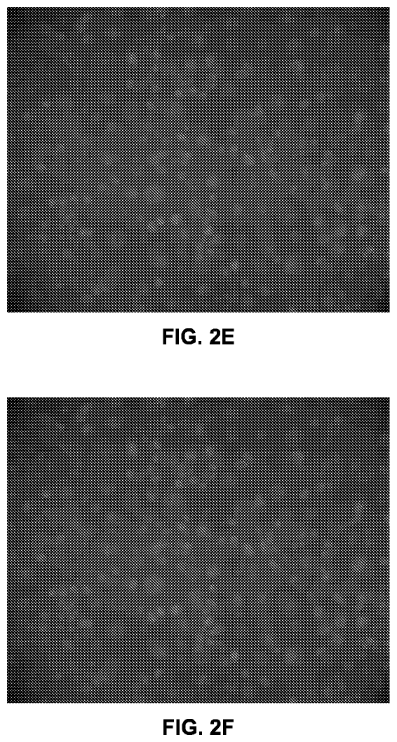

FIG. 2A illustrates the untreated cells after staining with the senescence β-galactosidase staining kit. FIG. 2B illustrates the untreated cells after staining with the anti-AGE antibody conjugated to GFP. FIG. 2C illustrates the untreated cells after staining with the anti-AGE antibody conjugated to GFP-DAPI. FIGS. 2B and 2C have been brightened to enhance contrast. The untreated cells appear relatively uniform in size and shape and are densely packed.

FIG. 2D illustrates the etoposide-treated cells after staining with the senescence β-galactosidase staining kit. FIG. 2E illustrates the etoposide-treated cells after staining with the anti-AGE antibody conjugated to GFP. FIG. 2F illustrates the etoposide-treated cells after staining with the anti-AGE antibody conjugated to GFP-DAPI. FIGS. 2E and 2F have been brightened to enhance contrast. The etoposide-treated cells have an irregular appearance, are larger in size and are loosely packed.

The results demonstrate that the administration of chemotherapeutic agents induces senescence in the PANG-1 cells. The results also confirm that anti-AGE antibodies bind to cells that have become senescent after exposure to chemotherapeutic agents.

### Example 18: Fluorescence Microscopy Study of Chemical Exposure-Induced Senescence

Cells from the human histiocytic lymphoma U937 cell line were treated with doxorubicin, a chemotherapeutic agent, to induce senescence. Cells were treated with 0 μM, 0.01 μM, 0.1 μM or 1 μM doxorubicin for 3 days, or were treated with 0 μM, 0.1 μM or 1 μM doxorubicin for 6 days. Senescence was measured with a senescence-associated β-galactosidase assay by fluorescent microscope imaging.

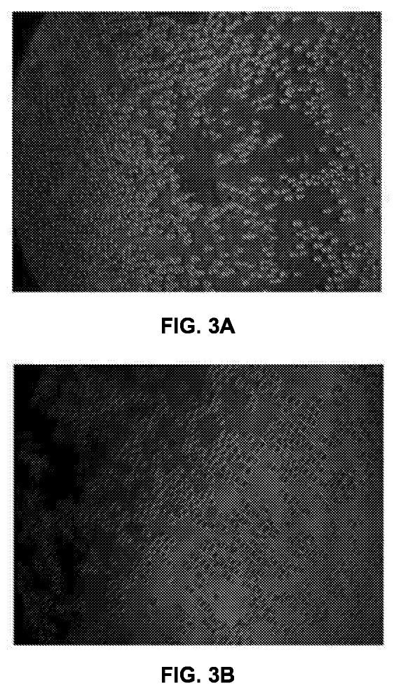

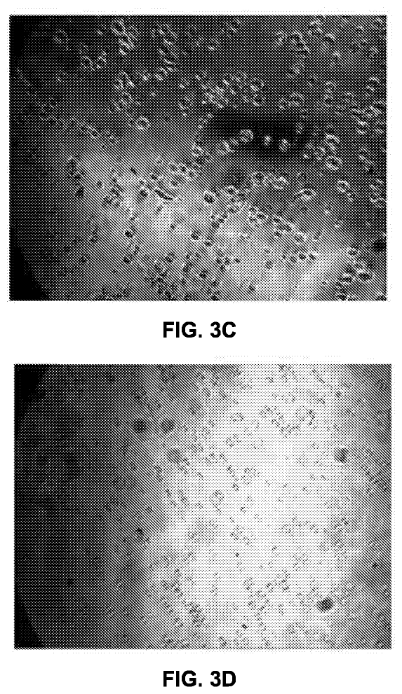

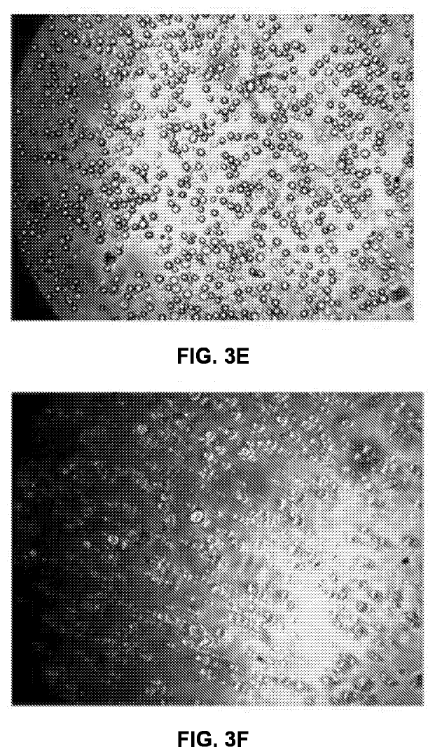

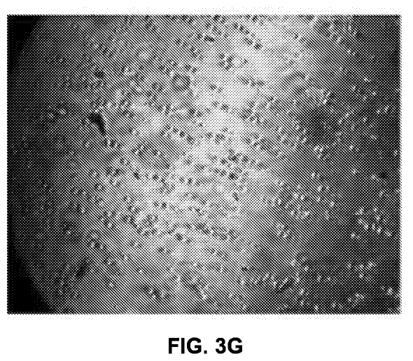

FIG. 3A-D illustrates the results of treating the cells with 0 μM (FIG. 3A), 0.01 μM (FIG. 3B), 0.1 μM (FIG. 3C) or 1 μM (FIG. 3D) doxorubicin for 3 days. At 0 μM doxorubicin <1% of cells fluoresce weakly. At 0.01 μM doxorubicin about 85% of cells fluoresce. At 0.1 μM doxorubicin about 65% of cells fluoresce. At 1 μM doxorubicin about 60% of cells fluoresce. FIG. 3E-G illustrates the results of treating the cells with 0 μM (FIG. 3E), 0.1 μM (FIG. 3F) or 1 μM (FIG. 3G) doxorubicin for 6 days. At 0 μM doxorubicin about 1% of cells fluoresce weakly. At 0.1 μM doxorubicin about 85% of cells fluoresce. At 1 μM doxorubicin about 85% of cells fluoresce. Treatment with 1 μM doxorubicin caused significant cell death. Peak senescence induction appears to be 0.1 μM doxorubicin treatment for 3-6 days.

The results demonstrate that the administration of chemotherapeutic agents induces senescence in the U937 cells.

### REFERENCES

1. “Progeroid syndromes”, available online at en.wikipedia.org/wiki/Progeroid_syndromes (Nov. 29, 2017).
2. Richardson, R. B., “Ionizing radiation and aging: rejuvenating an old idea”, Aging, Vol. 1, No. 11, pp. 887-902 (2009).
3. Meng, A. et al., “Ionizing radiation and Bisulfan induce premature senescence in murine bone marrow hematopoietic cells”, Cancer Research, Vol. 63, pp. 5414-5419 (2003).
4. Flament, F. et al., “Effect of the sun on visible clinical signs of aging in Caucasian skin”, Clinical, Cosmetic and Investigational Dermatology, Vol. 6, pp. 221-232 (2016).
5. Smith, R. L. et al., “Premature and accelerated aging: HIV or HAART?”, Frontiers in Genetics, Vol. 3, Article 328, pp. 1-10 (2013).
6. Cupit-Link, M. C. et al., “Biology of premature ageing in survivors of cancer”, ESMO Open, Vol. 2, No. e000250, pp. 1-9 (2017).
7. White, S. S. et al., “An overview of the effects of dioxins and dioxin-like compounds on vertebrates, as documented in human and ecological epidemiology”, Journal of Environmental Science and Health. Part C, Environmental Carcinogenesis & Ecotoxicology Reviews, Vol. 27, No. 4, pp. 197-211 (2009).
8. Ribas, J. et al., “Biomechanical strain exacerbates inflammation on a progeria-on-a-chip model”, Small, Vol. 13 (2017).
9. D'Orazio, J. et al., “UV radiation and the skin”, International Journal of Molecular Sciences, Vol. 14, pp. 12222-12248 (2013).
10. Roninson, I. B., “Tumor cell senescence in cancer treatment”, Cancer Research, Vol. 63, pp. 2705-2715 (2003).
11. Zhu, Y. et al., “The Achilles' heel of senescent cells: from transcriptome to senolytic drugs”, Aging Cell, Vol. 14, pp. 644-658 (2015).
12. Formenti, S. C. et al., “Combining radiotherapy and cancer immunotherapy: a paradigm shift”, Journal of the National Cancer Institute, Vol. 105, No. 4, pp. 256-265 (2013).
13. Pan, J. et al., “Inhibition of Bcl-2/xl with ABT-263 selectively kills senescent type II pneumocytes and reverses pulmonary fibrosis induced by ionizing radiation in mice”, International Journal of Radiation Oncology Biology Physics, Vol. 99, No. 2, pp. 353-361 (2017).
14. Palmer, J. L. et al., “Combined radiation and burn injury results in exaggerated early pulmonary inflammation”, Radiation Research, Vol. 180, No. 3, pp. 276-283 (2013).
15. Wilgus, T. A. et al., “Regulation of scar formation by vascular endothelial growth factor”, Laboratory Investigation, Vol. 88, pp. 579-590 (2008).
16. Shaw, J. N. et al., “N^{ε}-(carboxymethyl)lysine (CML) as a biomarker of oxidative stress in long-lived tissue proteins”, Oxidative Stress Biomarkers and Antioxidant Protocols, Chapter 15, Humana Press, pp. 129-137 (2002).
17. Leeman, K. T. et al., “Lung stem and progenitor cells in tissue homeostasis and disease”, Current Topics in Developmental Biology, Vol. 107, pp. 207-233 (2014).
18. Haddadi, G. H. et al., “Hesperidin as radioprotector against radiation-induced lung damage in rat: a histopathological study”, Journal of Medical Physics, Vol. 42, No. 1, pp. 25-32 (2017).
19. Brandl, A. et al., “Oxidative stress induces senescence in chondrocytes”, Journal of Orthopaedic Research, Vol. 29, pp. 1114-1120 (2011).

1. A method of treating or preventing a chronic effect of radiation exposure, comprising administering to a subject a composition comprising an anti-AGE antibody.

2. The method of claim 1, further comprising administering to the subject a composition comprising a second anti-AGE antibody;
wherein the second anti-AGE antibody is different from the first anti-AGE antibody.

3. The method of claim 1, further comprising:
testing the subject for effectiveness of the first administration at treating the chronic effect of radiation exposure; followed by
a second administering of the anti-AGE antibody.

4-6. (canceled)

7. A method of treating or preventing the onset of a chronic effect of radiation exposure, comprising immunizing a subject in need thereof against AGE-modified proteins or peptides of a cell.

8. The method of claim 7, wherein the immunizing comprises:
administering a first vaccine comprising a first AGE antigen; and
optionally, administering a second vaccine comprising a second AGE antigen;
wherein the second AGE antigen is different from the first AGE antigen.

9-10. (canceled)

11. A method of treating or preventing a chronic effect of chemical exposure, comprising administering to a subject a composition comprising an anti-AGE antibody.

12. The method of claim 11, further comprising administering to the subject a composition comprising a second anti-AGE antibody;
wherein the second anti-AGE antibody is different from the first anti-AGE antibody.

13. The method of claim 11, further comprising:
testing the subject for effectiveness of the first administration at treating the chronic effect of chemical exposure; followed by
a second administering of the anti-AGE antibody.

14-16. (canceled)

17. A method of treating or preventing the onset of chronic effect of chemical exposure, comprising immunizing a subject in need thereof against AGE-modified proteins or peptides of a cell.

18. The method of claim 17, wherein the immunizing comprises:
administering a first vaccine comprising a first AGE antigen; and
optionally, administering a second vaccine comprising a second AGE antigen;
wherein the second AGE antigen is different from the first AGE antigen.

19-22. (canceled)

23. The method of claim 1, wherein the subject is a human.

24-26. (canceled)

27. The method of claim 1, wherein the anti-AGE antibody binds an AGE antigen comprising at least one protein or peptide that exhibits AGE modifications selected from the group consisting of FFI, pyrraline, AFGP, ALI, carboxymethyllysine, carboxyethyllysine and pentosidine.

28. The method of claim 1, wherein the anti-AGE antibody binds a carboxymethyllysine-modified protein or peptide.

29-36. (canceled)

37. The method of claim 8, wherein the first and second AGE antigens are each independently an AGE-modified protein or peptide selected from the group consisting of AGE-RNAse, AGE-human hemoglobin, AGE-albumin, AGE-BSA, AGE-human serum albumin, AGE-ovalbumin, AGE-low density lipoprotein, AGE-collagen IV, AGE-antithrombin III, AGE-calmodulin, AGE-insulin, AGE-ceruloplasmin, AGE-collagen, AGE-cathepsin B, AGE-albumin, AGE-crystallin, AGE-plasminogen activator, AGE-endothelial plasma membrane protein, AGE-aldehyde reductase, AGE-transferrin, AGE-fibrin, AGE-copper/zinc SOD, AGE-apo B, AGE-fibronectin, AGE-pancreatic ribose, AGE-apo A-I and II, AGE-hemoglobin, AGE-Na⁺/K⁺-ATPase, AGE-plasminogen, AGE-myelin, AGE-lysozyme, AGE-immunoglobulin, AGE-red cell Glu transport protein, AGE-β-N-acetyl hexominase, AGE-apo E, AGE-red cell membrane protein, AGE-aldose reductase, AGE-ferritin, AGE-red cell spectrin, AGE-alcohol dehydrogenase, AGE-haptoglobin, AGE-tubulin, AGE-thyroid hormone, AGE-fibrinogen, AGE-β₂-microglobulin, AGE-sorbitol dehydrogenase, AGE-α₁-antitrypsin, AGE-carbonate dehydratase, AGE-RNAse, AGE-low density lipoprotein, AGE-hexokinase, AGE-apo C-I, AGE-KLH and mixtures thereof.

38. (canceled)

39. The method of claim 8, wherein the first AGE antigen comprises a carboxymethyllysine-modified protein or peptide.

40-47. (canceled)

48. The method of claim 7, further comprising testing the patient to determine if the chronic effect has been ameliorated, and
repeating the immunizing, if necessary.

49-57. (canceled)

58. The method of claim 1, wherein the antibody has a rate of dissociation (k_{d}) of at most 9×10⁻³ sec⁻¹.

59-61. (canceled)

62. The method of claim 1, wherein the chronic effect of radiation exposure comprises at least one symptom which mimics premature aging selected from the group consisting of gray hair, wrinkles, frailty, cataracts, arteriosclerosis, atherosclerosis, Alzheimer's disease, Parkinson's disease, sarcopenia, loss of adipose tissue, lordokyphosis, cancer, premature menopause, cardiovascular disease, dementia, Type II diabetes, endocrinopathies, cardiac dysfunction, osteoporosis, osteoarthritis, pulmonary fibrosis, kidney and liver disease, metabolic disorders, lipodystrophy, hearing loss, vision loss and memory loss.

63-64. (canceled)

65. The method of claim 1, wherein the radiation comprises at least one type of radiation selected from the group consisting of alpha radiation, beta radiation, gamma radiation, X-ray radiation, and neutron radiation.

66. The method of claim 11, wherein the chemical exposure comprises exposure to a chemical weapon, a chemotherapy agent, a highly active antiretroviral therapy (HAART) agent, a poison, or an oxidizing agent.

67-69. (canceled)
